(19) United States
(12) Patent Application Publication
(10) Pub. No.: US 2025/0025584 A1
(43) Pub. Date: Jan. 23, 2025

(54) RADIOPHARMACEUTICAL TREATMENT METHODS AND USE

(71) Applicant: POINT BIOPHARMA, INC., Indianapolis, IN (US)

(72) Inventors: Neil FLESHNER, Indianapolis, IN (US); Jessica JENSEN, Indianapolis, IN (US); Joe MCCANN, Toronto (CA)

(21) Appl. No.: 18/675,346

(22) Filed: May 28, 2024

Related U.S. Application Data
(63) Continuation of application No. PCT/US22/80572, filed on Nov. 29, 2022.
(60) Provisional application No. 63/283,999, filed on Nov. 29, 2021.

(51) Int. Cl. A61K 51/08; A61P 35/00

(52) U.S. Cl. CPC A61K 51/088; A61P 35/00; A61K 2121/00

(57) ABSTRACT

In one aspect, therapeutic use, methods of treatment and kits comprising ¹⁷⁷Lu-PSMA I&T are provided for subjects exhibiting prostate cancer progression following administration with one or more androgen receptor inhibitor agents.

### CROSS REFERENCE TO RELATED APPLICATION

This application is a continuation of International Application No. PCT/US2022/080572, which designated the United States and was filed on Nov. 29, 2022, which claims the benefit of U.S. Provisional Application 63/283,999 filed on Nov. 29, 2021. The entire teachings of the above applications are incorporated herein by reference.

### FIELD

In one aspect, therapeutic use and methods of treatment comprising ¹⁷⁷Lu-PSMA I&T are provided for subjects exhibiting prostate cancer progression following administration with one or more androgen receptor inhibitor agents.

### BACKGROUND

Radiopharmaceuticals have been used for a variety of therapeutic and diagnostic indications. Among others, radiolabeled molecules have been useful to treat various malignant tumors.

Certain androgen receptor inhibitors have been utilized or treatment of prostate cancer. These agents are not, however, consistently effective. See, for instance, Watson et al., Nature Reviews Cancer, volume 15, pages 701-711 (2015).

It thus would be desirable to have new anti-cancer therapies. It would be particularly desirable to have new therapies that would be utilized against tumors that are not effectively treated with androgen receptor inhibitors.

### SUMMARY

We now provide new therapeutic uses of a complex of lutetium-177 and EuK-Sub-kf-iodo-α-DOTAGA (¹⁷⁷Lu-PSMA I&T).

In particular aspects, methods are provided for treating a subject suffering from cancer, comprising a) identifying a subject exhibiting cancer progression during treatment with one or more androgen receptor inhibitor agents; and b) administering to the identified subject an effective amount of a complex of lutetium-177 and EuK-Sub-kf-iodo-y-DOTAGA.

Methods are also provided for treating a subject suffering from cancer, comprising a) administering to the subject a therapeutically effective amounts of one or more androgen receptor inhibitor agents; b) identifying the subject as exhibiting cancer progression following administering the one or more androgen reception inhibitor agents; and thereafter c) administering to the identified subject an effective amount of a complex of lutetium-177 and EuK-Sub-kf-iodo-y-DOTAGA to patient.

In further aspects, methods are provided for treating a subject suffering from prostate cancer, comprising: a) identifying a subject i) exhibiting prostate cancer progression during or after treatment with one or more androgen receptor inhibitor agents, and ii) being chemotherapy-naïve before treatment with one or more androgen receptor inhibitor agents; b) administering to the identified subject an amount of a lutetium-177 labeled PSMA-targeted radioligand agent to provide from a 4 to 10 Gbq dose, which subject is dosed at least four times, at four week or longer intervals between doses, with the lutetium-177 labeled PSMA-targeted radioligand agent. In preferred aspects, the lutetium-177 labeled PSMA-targeted radioligand agent is a complex of lutetium-177 and EuK-Sub-kf-iodo-y-DOTAGA, particularly ¹⁷⁷Lu-PSMA I&T.

In one aspect of the above methods, the subject has exhibited cancer progression during treatment with one or more of abitaterone, enzalutamide, apalutamide, darolutamide, cimetidine, orteronel, galeterone, sevitronel, topilutamide, bicalutamide, fluamide and/or nilutamide.

In a further aspect, the subject has exhibited cancer progression during treatment with abitaterone, enzalutamide, apalutamide and/or darolutamide.

In certain aspects, the subject will be chemotherapy-naïve before administration of any androgen receptor inhibitor agents, and other than the androgen receptor inhibitor therapy, the subject will be chemotherapy-naïve before administration of ¹⁷⁷Lu-PSMA I&T, i.e. the subject will not have received any other chemotherapy (e.g. docetaxel-based chemotherapy or other radiopharmaceutical therapy) for treatment of the subject's cancer.

The subject may be treated with such agents for varying times and under varying conditions. Thus, in certain aspects, the subject may be exhibited cancer progression following treatment with one or more androgen receptor inhibitor agents for up to 1, 2, 3 or 4 weeks, or longer periods such as up to 2, 3, 4, 5, 6 months or more.

In certain aspects, the subject may be assessed for and identified as exhibiting high or elevated prostate specific membrane antigen (PSMA) expression relative to a healthy subject and that identified subject with elevated PSMA activity is administered a complex of lutetium-177 and EuK-Sub-kf-iodo-y-DOTAGA.

In certain aspects, the present methods and uses include imaging or other assessment of the subject's cancer, including an assessment of PSMA expression. That assessment suitably may occur before administering one or more androgen receptor inhibitor agents and/or before administering a complex of lutetium-177 and EuK-Sub-kf-iodo-y-DOTAGA (¹⁷⁷Lu-PSMA I&T). The cancer assessment preferably includes imaging, for example, SPECT or PET imaging, or immunohistochemistry (IHC) or fluorescence in situ hybridization (FISH) imaging. An imaging agent such as the ¹⁸F or ⁶⁸Ga agent (such as agents disclosed in Example 2 below), or ⁶⁴Cu agents can be administered to the subject to facilitate the cancer analysis.

In one aspect, a subject is identified for treatment where the subject exhibits a PSMA expression standard uptake value of 1) (SUV)max≥15 at one site of disease and/or 2) SUVmax>10 at all measurable disease sites.

In another aspect, a subject is identified for treatment where the subject exhibits at least 1 positive lesion SUVmax>10.

In a further aspect, a subject is identified for treatment where the subject has an elevated prostate specific antigen (PSA) level, for example a PSA level of 3 ng/mL, 4 ng/mL, 5 ng/mL or 6 ng/mL or higher.

In certain embodiments, administration of one or more androgen receptor inhibitor agents is terminated before administering the complex of lutetium-177 and EuK-Sub-kf-iodo-y-DOTAGA.

In certain aspects, the subject is suffering from a prostate specific membrane antigen (PSMA) expressing cancer. In certain aspects, the subject has prostate cancer. In particular aspects, the subject is suffering from metastatic castration-resistant prostate cancer (mCRPC). In additional aspects, the subject is suffering from metastatic castration-resistant prostate cancer (mCRPC) with prostate-specific membrane antigen (PSMA)-avid lesions.

In certain aspects, the complex of lutetium-177 and EuK-Sub-kf-iodo-y-DOTAGA (¹⁷⁷Lu-PSMA I&T) is administered parenterally, such as intradermal, subcutaneous, intramuscular, intraperitoneal and/or intravenous. In certain methods, ¹⁷⁷Lu-PSMA I&T is administered in a dosage amount (single administration amount or single day amount) of about 1 GBq to about 15 GBq, or about 2 GBq to about 14 GBq, or about 3 or 4 GBq to about 10 or 12 GBq, or about 5 or 6 GBq to about 7, 8 or 9 GBq. A dosage of 6.8 GBq+/−10% per administration may be particularly preferred for various treatments. ¹⁷⁷Lu-PSMA I&T suitably may be administered under varying schedules including multiple times per week, or once every 1, 2, 3, 4, 5, 6, 7, 8, 9, 10, 11 or 12 or more weeks. In some aspects, scheduled ¹⁷⁷Lu-PSMA I&T administration may continue for up to 2, 3, 4, 5, 6, 7, 8, 9, 10, 12, 14, 16, 18, 20, 24, 26, 28 or 30 months.

¹⁷⁷Lu-PSMA I&T is a complex of lutetium (¹⁷⁷Lu) and EuK-Sub-kf-iodo-y-DOTAGA. The term “complex” herein generally refers to a union of ¹⁷⁷Lu and the ligand EuK-Sub-kf-iodo-y-DOTAGA inclusive of chemical and physical variations that may exist with the joined or associated lutetium-177 and EuK-Sub-kf-iodo-y-DOTAGA (designated as ¹⁷⁷Lu-PSMA I&T). Also, ¹⁷⁷Lu-PSMA I&T and 177Lu-PNT2002 are used interchangeably herein and have the same meaning.

¹⁷⁷Lu-PSMA I&T has several possible stereoisomers, including the R and S isomers of the carbon that is an N-ring substituent of the tetraazacyclotetradecane moiety of the compound. References herein to EuK-Sub-kf-iodo-y-DOTAGA and ¹⁷⁷Lu-PSMA I&T without further limitation includes all possible stereoisomers of each of those compounds and particularly both the noted R and S isomers.

In certain aspects, racemic mixtures of ¹⁷⁷Lu-PSMA I&T are utilized in the present pharmaceutical compositions and methods.

In other preferred aspects, optically enriched mixtures of ¹⁷⁷Lu-PSMA I&T and EuK-Sub-kf-iodo-y-DOTAGA are used in the present methods and pharmaceutical compositions.

In certain preferred aspects, the R isomer of ¹⁷⁷Lu-PSMA I&T is provided, including for use in the present pharmaceutical compositions and methods. That R isomer may be represented by the following structure 2A:

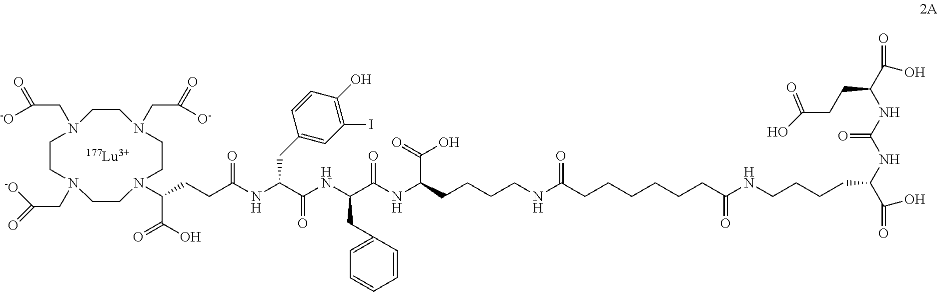

In one aspect, for use in the present methods and pharmaceutical compositions, generally preferred is ¹⁷⁷Lu-PSMA I&T that is substantially optically enriched with the R isomer of structure 2A, or is an enantiomerically pure mixture of the R isomer of structure 2A.

In another aspect, the S isomer of ¹⁷⁷Lu-PSMA I&T is used in the present pharmaceutical compositions and methods. That S isomer may be represented by the following structure 2B:

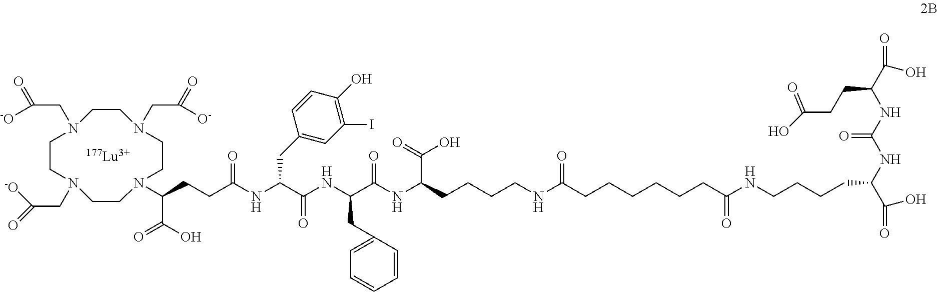

In one aspect, for use in the present methods and pharmaceutical compositions, generally preferred is ¹⁷⁷Lu-PSMA I&T that is substantially optically enriched with the S isomer of structure 2B, or is an enantiomerically pure mixture of the S isomer of structure 2B.

¹⁷⁷Lu-PSMA I&T for use in the present methods preferably has high chemical or radiochemical purities, including greater than 95, 96, 97, 98, 99, 99.5, 99.6, 99.7, 99.8 or 99.9 percent radiochemical purity and/or substantial absence of one or more prior impurities.

In further aspects, treatment kits are provided. In a preferred aspect, a kit may comprise a) a pharmaceutical preparation of lutetium-177 labeled PSMA-targeted radioligand agent suitable for intravenous administration to a patient, and in an amount sufficient to provide from a 4 to 10 Gbq dose; and b) a package insert containing instructions for use of the agent in the treatment of a subject exhibiting prostate-specific membrane antigen (PSMA)-positive metastatic castration-resistant prostate cancer (mCRPC) who have previously been treated with androgen receptor (AR) pathway inhibition but which patient is taxane chemotherapy-naïve or taxane chemotherapy adverse (i.e., ineligible or averse to chemotherapeutic treatment options).

Uses of a lutetium-177 labeled PSMA-targeted radioligand agent to treat a subject (such as human) suffering from cancer including prostate cancer are also provided, wherein a) a subject is identified as i) exhibiting prostate cancer progression during or after treatment with one or more androgen receptor inhibitor agents, and ii) being chemotherapy-naïve before treatment with the one or more androgen receptor inhibitor agents; b) an amount of a lutetium-177 labeled PSMA-targeted radioligand agent is administered providing from a 4 to 10 Gbq dose, which subject is dosed at least four times, at four week or longer intervals between doses, with the lutetium-177 labeled PSMA-targeted radioligand therapy. In preferred aspects, the lutetium-177 labeled PSMA-targeted radioligand agent is a complex of lutetium-177 and EuK-Sub-kf-iodo-y-DOTAGA.

Other aspects of the invention are disclosed infra.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein as a shorthand notation to represent the expression “and,” describing the combination of items, as well as “or,” describing the items in the alternative form.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

The term “about”, as used herein, means an acceptable margin of error for a particular value, which depends in part on how the value is measured or determined. In certain embodiments, “about” as used herein will be understood by persons of ordinary skill in the art to mean up to plus or minus 20% of the particular term. In further embodiments, “about” as used herein will be understood by persons of ordinary skill in the art to mean up to plus or minus 10% of the particular term.

As used herein, the term “optically enriched” or “optical excess” denotes the presence of one or more non-racemic stereoisomeric centers in a molecule, wherein the configuration of at least one stereoisomeric center has a predominance of one stereoisomeric configuration (R or S). For example, one stereoisomeric center in a molecule, typically a carbon atom, may have greater than 50 or 55 weight % (based on total weight of the compound) of its attached atoms spatially arranged in the (R) configuration. Alternatively, more than 50 weight % (based on total weight of the compound) may be spatially arranged in the (S) configuration. More preferably the molecule, or its stereoisomeric center, is substantially optically enriched, and even more preferably is substantially enantiomerically pure.

As used herein, the term “substantially optically enriched” or “substantial optical excess”, when referring to a stereoisomer or stereoisomeric center, denotes that at least about 60 weight % (based on total weight of the compound), preferably about 70 weight % (based on total weight of the compound), more preferably about 80 weight % (based on total weight of the compound), still more preferably about 90 weight % (based on total weight of the compound) of one stereoisomer or one stereoisomeric center configuration predominates in the mixture, with at least about 95 weight % (based on total weight of the compound) of one stereoisomer or one stereoisomeric center configuration being even more preferred. In some preferred embodiments, the compound is “substantially enantiomerically pure”, that is, at least about 97.5 weight % (based on total weight of the compound), more preferably about 99 weight % (based on total weight of the compound), even more preferably about 99.5 weight % (based on total weight of the compound) of one stereoisomeric configuration predominates.

As used herein, the term “substantially pure” means sufficiently homogeneous to appear free of readily detectable impurities as determined by standard analytical methods, such as thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” refers to a collection of molecules, wherein at least about 55%, about 60%, about 65%, about 70%, about 75%, about 80%, about 85%, about 90%, about 95%, about 97%, about 98%, about 98.5%, about 99%, about 99.5% or about 99.9% or greater of the molecules are a single compound, including a racemic mixture or a single stereoisomer thereof, as determined by standard analytical methods.

As used herein, and unless otherwise specified, the terms “treat,” “treating” and “treatment” refer to the eradication or amelioration of a disease, disorder, or condition, or of one or more symptoms associated with the disease, disorder or condition. In certain embodiments, the terms refer to minimizing the advancement or worsening of the disease, disorder, or condition resulting from the administration of a formulation of the invention to a patient with such a disease, disorder, or condition. In some embodiments, the terms refer to the administration of a formulation provided herein, after the onset of symptoms of the particular disease, disorder, or condition. The terms “treat,” “treating”, “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition. In a particular embodiment the terms “treat,” “treating”, “treatment”, or the like, covers the treatment of a disease, disorder, or condition in a mammal, e.g., a primate, e.g., a human, and includes at least one of (i), (ii), and (iii) above. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the terms “subject”, and “patient” are used interchangeably. The terms “subject” and “patient” refer to an animal such as a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

### BRIEF DESCRIPTION OF THE DRAWINGS

FIG. 1: Demographics and baseline characteristics of Safety Population (n=27).

FIG. 2: Treatment exposure of Safety Population (n=27).

FIG. 3: Kaplan-Meier curve for radiographic progression-free survival. rPFS follow-up calculated as time in months from date of enrollment to date of last available imaging (conventional) prior to the data cut-off for all enrolled participants.

FIG. 4: Complete response participant vignette.

FIG. 5: Overall incidence of treatment-emergent adverse events (TEAEs), all grades.

FIGS. 6A-C: Administration techniques. (6A) Gravity method. (6B) Pump method with vial. (6C) Pump method with syringe.

### DETAILED DESCRIPTION

In one preferred aspect, we now provide methods for treatment of patients suffering from prostate cancer, including metastatic castration-resistant prostate cancer (mCRPC).

More specifically, patients with prostate cancer such as mCRPC may undergo prostate-specific membrane-antigen (PSMA)-based analysis such as PET or other imaging to identify and select the patient for treatment with ¹⁷⁷Lu-PSMA I&T. ¹⁷⁷Lu-PSMA I&T then may be administered to selected patients, including following androgen receptor-axis-targeted therapy (ARAT).

In one aspect, high PSMA avidity or expression in a patient may be assessed by the following 1) or 2):
1) For a patient with measurable disease (using Response Evaluation Criteria in Solid Tumors (RECIST) 1.1 criteria):
i) Threshold standard uptake value (SUV)max≥15 at one site of disease, and
ii) SUVmax>10 at all measurable disease sites.
2) For a patient without measurable disease, at least 1 positive lesion SUVmax>10.

As referred to herein, SUV (standard uptake value) may be assessed by known procedures, such as administering a ⁶⁸Ga or ¹⁸F compound or other tracer and imaging (e.g. PET, PET/CT) the subject. See Dietlein et al. Mol. Imaging Biol. 2015; 17(4):575-584 for SUV analysis and determination of values in a subject. See also Giesel et al., J Nucl Med. 2018; 59(7):1076-1080 for SUV (including SUVmax) analysis and determination of values in a subject that can be utilized for determination of SUV and SUVmax as referred to herein.

### ¹⁷⁷Lu-PSMA I&T

¹⁷⁷Lu-PSMA I&T (including structures 2A and/or 2B below) can be prepared by complexing or incorporating ¹⁷⁷Lu (lutetium-177) or halide thereof such as ¹⁷⁷LuCl₃ with EuK-Sub-kf-iodo-y-DOTAGA (structures 1A and/or 1B below).

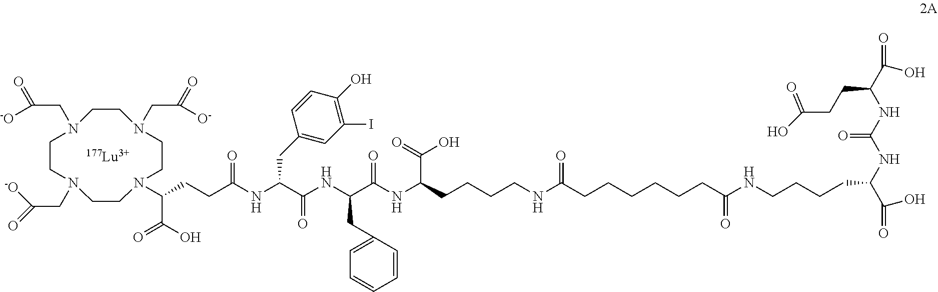

An IUPAC designation of structure 2A is lutetate(5-)-177Lu, [N-[(4S)-4-(carboxy-KO)-4-[4,7,10-tris[(carboxy-KO)methyl]-1,4,7,10-tetraazacyclododec-1-yl-KN1,KN4,KN7,KN10]-1-oxobutyl]-3-iodo-D-tyrosyl-D-phenylalanyl-N6-[8-[[(5S)-5-carboxy-5-[[[[[(1S)-1,3-dicarboxypropyl]amino]carbonyl]amino]pentyl]amino]-1,8-dioxooctyl]-D-lysinato(8-)]-, hydrogen (1:5).

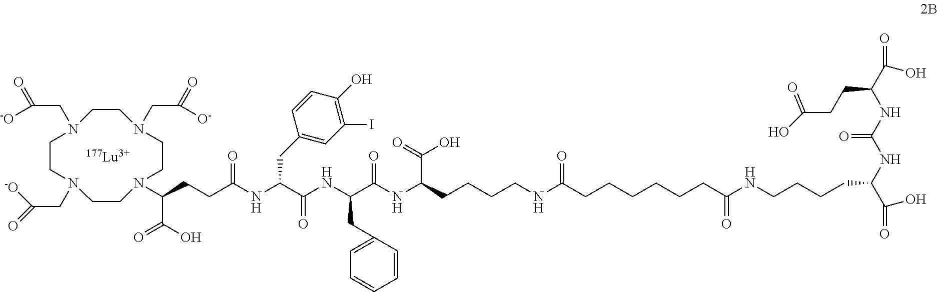

An IUPAC designation of compound 2B is lutetate(5-)-177Lu,N-[(4R)-4-(carboxy-KO)-4-[4,7,10-tris[(carboxy-KO)methyl]-1,4,7,10-tetraazacyclododec-1-yl-KN1,KN4,KN7,KN10]-1-oxobutyl]-3-iodo-D-tyrosyl-D-phenylalanyl-N6-[8-[[(5S)-5-carboxy-5-[[[[(1S)-1,3-dicarboxypropyl]amino]carbonyl]amino]pentyl]amino]-1,8-dioxooctyl]-D-lysinato(8-)]-, hydrogen (1:5).

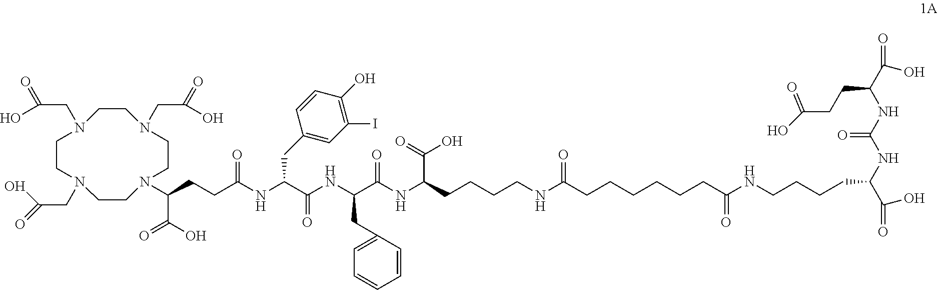 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CC[C@@H](C(=O)O)N1CCN(CC(=O)O)CCN(CC(=O)O)CCN(CC(=O)O)CC1)C(=O)O)C(=O)O)C(=O)O]

The S isomer compound of structure 1A has an IUPAC name of N-[(4S)-4-(carboxy-KO)-4-[4,7,10-tris[(carboxy-KO)methyl]-1,4,7,10-tetraazacyclododec-1-yl-KN1,KN4,KN7,KN10]-1-oxobutyl]-3-iodo-D-tyrosyl-D-phenylalanyl-N6-[8-[[(5S)-5-carboxy-5-[[[[(1(S)-1,3-dicarboxypropyl]amino]carbonyl]amino]pentyl]amino]-1,8-dioxooctyl]-D-lysinato(8-)]-, hydrogen (1:5)).

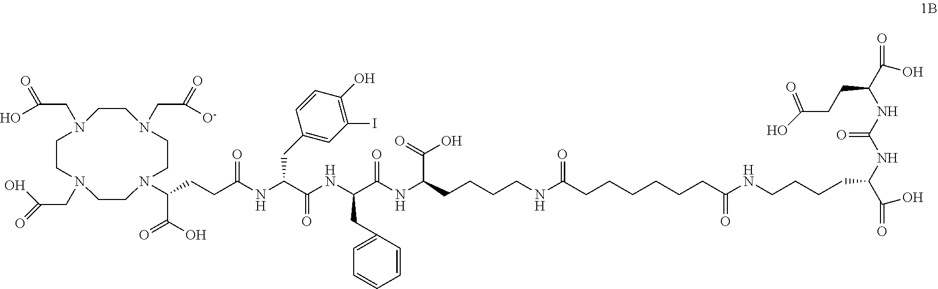 [SMILES: O=C([O-])CN1CCN(CC(=O)O)CCN(CC(=O)O)CCN([C@H](CCC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)C(=O)O)CC1]

The R isomer compound of structure 1B has an IUPAC name of: [N-[(4R)-4-(carboxy-KO)-4-[4,7,10-tris[(carboxy-KO)methyl]-1,4,7,10-tetraazacyclododec-1-yl-KN1,KN4,KN7,KN10]-1-oxobutyl]-3-iodo-D-tyrosyl-D-phenylalanyl-N6-[8-[[(5S)-5-carboxy-5-[[[[(1S)-1,3-dicarboxypropyl]amino]carbonyl]amino]pentyl]amino]-1,8-dioxooctyl]-D-lysinato(8-)]-, hydrogen (1:5)).

The structures 1A and/or 1B may be suitably formed as described in Weineisen et al. J Nucl Med 2015; 56:1169-1176; and Chatalic, Theranostics, 6(6), 849-861 (2016). To provide an optically enriched or substantially optically enriched or enantiomerically pure sample of ¹⁷⁷Lu-PSMA I&T the corresponding optical isomer of EuK-Sub-kf-iodo-y-DOTAGA may be used in the incorporation reaction. That is, the compound of structure 1A may be reacted with lutetium-177 to provide the S isomer complex of structure 2A, and the compound of structure 1B may be reacted with lutetium-177 to provide the R isomer complex of structure 2B. The structures 1A and/or 1B also are available from piCHEM (RaabaGrambach, Austria). Optically enriched mixtures of structures 1A and/or 1B suitably may be prepared with use of an optically enriched precursor (reagent) and/or separation of optical isomers with an appropriate optically active reagent such as an optically active salt.

It is understood that ¹⁷⁷Lu-PSMA I&T as referred to herein includes the above structures 2A and/or 2B as well as other complexes of lutetium (¹⁷⁷Lu) and EuK-Sub-kf-iodo-y-DOTAGA. For instance, references herein to ¹⁷⁷Lu-PSMA I&T include compounds that generally correspond to structure 2A and/or 2B but where the ¹⁷⁷Lu substantially complexes to other portions or moieties (such as one or more other nitrogens) of the EuK-Sub-kf-iodo-y-DOTAGA molecule than as depicted in structures 2A and 2B above. References to ¹⁷⁷Lu-PSMA I&T also may include other stereoisomers than those shown in structures 1A, 1B, 2A, and 2B above, although the stereoisomers depicted in structures 1A, 1B, 2A, and 2B are preferred, particularly structures 1A and 2A.

To synthesize ¹⁷⁷Lu-PSMA I&T, lutetium-177 (¹⁷⁷Lu) can be admixed with EuK-Sub-kf-iodo-y-DOTAGA. The ¹⁷⁷Lu suitably may be carrier added or more preferably no-carrier-added (n.c.a.) lutetium-177. To facilitate incorporation (e.g. complexing including chelating) of lutetium-177 with the EuK-Sub-kf-iodo-y-DOTAGA compound, preferably an admixture of the compounds is thermally treated.

### Pharmaceutical Compositions

¹⁷⁷Lu-PSMA I&T is suitably administered to a subject an aqueous composition including 1) a complex of lutetium-177 and EuK-Sub-kf-iodo-y-DOTAGA and 2) one or more stabilizer compounds such as one or more ascorbate compounds. Preferably, the radiochemical purity of the composition is at least 95%, 96%, 97%, 95% or 99% where the composition is maintained at 30° C. or less and for 3, 4 or 5 days or more following preparation of the composition.

In certain preferred embodiments, the pharmaceutical composition is free of unchelated lutetium-177 in an amount of not more than 2, 1.5, 1.0 or 0.5 weight % based on total weight of the pharmaceutical composition, such as may be determined by radiometric detection (including HPLC radiometric detection), where the composition is maintained at 30° C. or less and such purity levels are exhibited for 3, 4 or 5 days or more following preparation of the composition.

In additional preferred embodiments, the pharmaceutical composition is free of radiochemical impurities in an amount of not more than 5, 4, 3.5, 3, 2.5, 2, 1.5, 1 or 0.5 weight % based on total weight of the pharmaceutical composition, such as may be determined by radiometric detection (including HPLC radiometric detection), where the composition is maintained at 30° C. or less and such purity levels are exhibited for 3, 4 or 5 days or more following preparation of the composition.

In yet still additional preferred embodiments, the pharmaceutical composition is free of chemical impurities in an amount of not more than 5, 4, 3, 2, 1 or 0.5 weight % based on total weight of the pharmaceutical composition, such as may be determined by chromatography or other method including HPLC or HPLC/UV analysis, where the composition is maintained at 30° C. or less and such purity levels are exhibited for 3, 4 or 5 days or more following preparation of the composition.

In yet still additional preferred embodiments, the pharmaceutical composition is 1) free of unchelated lutetium-177 in an amount of not more than 2, 1.5, 1.0 or 0.5 weight % (such as may be determined by radiometric detection (including HPLC radiometric detection)); 2) free of radiochemical impurities in an amount of not more than 5, 4, 3.5, 3, 2.5, 2, 1.5, 1 or 0.5 weight % (such as may be determined by radiometric detection (including HPLC radiometric detection); and 3) free of chemical impurities in an amount of not more than 5, 4, 3, 2, 1 or 0.5 weight % (such as may be determined by HPLC/UV analysis), with all weight % based on total weight of the pharmaceutical composition, and where the composition is maintained at 30° C. or less and such purity levels are exhibited for 3, 4 or 5 days or more following preparation of the composition.

In certain embodiments, the pharmaceutical composition is formulated for parenteral administration, such as intravenous, intramuscular, intradermal, subcutaneous, intrathecal or intraperitoneal administration. For example, the pharmaceutical composition is formulated for intravenous, intramuscular, subcutaneous or intradermal injection. In preferred aspects, the pharmaceutical composition is formulated for intravenous administration. In typical embodiments, the pharmaceutical composition may be administered in a form of a pharmaceutical aqueous solution.

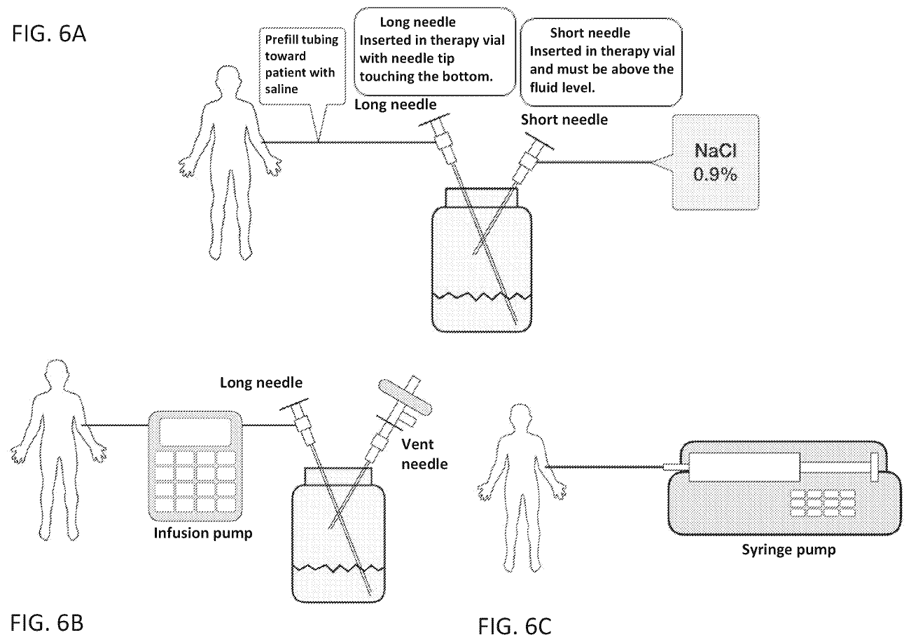

Because of the radioactive nature to the present drug compositions, injection and/or infusion of the drug to patients will typically require certain additional care to avoid exposure of the healthcare provider to radiation or contamination of the infusion facility. In certain embodiments, the pharmaceutical composition is provided in a vial and injected by intravenous push, such as using a disposable syringe, optionally with syringe shield if handheld infusion administration. In such instances, the desired radioactivity dose is withdrawn from vial using a syringe fitted with a syringe shield and disposable sterile needle and the pharmaceutical composition is administered by slow intravenous push (1-20 minutes, more preferably about 10 minutes) manually or with a syringe pump (FIG. 6C). A 3-way stopcock can optionally be used, to permit each of switching between preadministration saline flush and injection of drug composition.

FIGS. 6A and 6B illustrate other methods for intravenous injection directly from the vial in which the drug composition is provided. For instance, as shown in FIG. 6A, drug composition can be administered using a gravity fed technique in which saline (shown, or other pharmaceutically acceptable and injectable buffer) is feed from a higher position into the vial by a short needle and catheter during infusion. The short needle, connected to the saline or other infusion liquid, is placed into the vial but without touching the solution of drug composition in the vial. The long needle is inserted and pushed into the vial so to be in fluid communication with the drug composition. In other embodiments, such as shown in FIG. 6B, the short needle is instead a vented needles, and the long needle is connected to an infusion pump that pumps the drug composition from the vial into the patient being infused.

In certain embodiments of the packaged pharmaceutical kit of the present invention there are included directions including one or more of the above techniques, i.e., instructions for intravenous push injection (manual or by pump), gravity fed infusion and/or infusion pump.

In certain embodiments, the packaged pharmaceutical kit includes a disposable syringe, and more preferably includes a disposable syringe with a syringe radiation shield, such as made of lead or tungsten.

In certain embodiments, the pharmaceutical composition is an aqueous solution, dispersion or other admixture such as for injection and comprises ¹⁷⁷Lu-PSMA I&T (including ¹⁷⁷Lu-PSMA I&T having an optical excess of structure 2A) and preferably one or more ascorbate compounds. In further preferred embodiments, the pharmaceutical composition is an aqueous solution, dispersion or other admixture such as for injection and comprises 1)¹⁷⁷Lu-PSMA I&T including ¹⁷⁷Lu-PSMA I&T having an optical excess of structure 2A) and preferably one or more ascorbate compounds and one or more gentisate compounds.

In certain preferred embodiments, a pharmaceutical aqueous solution, dispersion or admixture may be utilized that includes: 1) a complex of lutetium-177 and EuK-Sub-kf-iodo-y-DOTAGA; and 2) at least one stabilizer compound that preferably can inhibit radiolytic degradation of the composition during storage following preparation of the complex. ¹⁷⁷Lu-PSMA I&T is suitably present in a concentration that it provides a volumetric radioactivity of at least 100 MBq/mL, preferably of at least 250, 500, 750 or 1000 MBq/mL within 1, 2, 3, 4 or 5 days following preparation. In certain aspects, ¹⁷⁷Lu-PSMA I&T is present in a concentration that it provides a volumetric radioactivity of from 100 to 1000 MBq/mL, preferably from or up to about 250, 500, 750 or 1000 MBq/mL within 1, 2, 3, 4 or 5 days following preparation.

In certain aspects, the pharmaceutical aqueous formulation has a shelf life of at least 24 hours at about 30° C. or less, at least 48 hours at about 30° C. or less, at least 72 hours at 30° C. or less, or from 24 hours to 120 hours at 30° C. or less, from 24 hours to 96 hours at 30° C. or less, from 24 hours to 84 hours at 30° C. or less, from 24 hours to 72 hours at 30° C. or less, in particular a shelf life of at least 72 hours at 30° C. or less. In further particular aspects, the pharmaceutical aqueous formulation has a shelf life of at least 96 hours (or 4 days) at about 30° C. or less, or the pharmaceutical aqueous formulation has a shelf life of at least 120 hours (or 5 days) at about 30° C. or less, or the pharmaceutical aqueous formulation has a shelf life of at least 144 hours (or 6 days) at about 30° C. or less.

In a particularly preferred aspect, ¹⁷⁷Lu-PSMA I&T is provided as a sterile solution for intravenous use. The ¹⁷⁷Lu-PSMA I&T solution suitably may be clear, colorless to slightly yellow. A single-dose vial suitably will contain 6.8+/−10% GBq ¹⁷⁷Lu-PSMA I&T for example calibrated at 1, 2, 3, 4, 5 or 6 or more days post-day of manufacture in 10 to 14 mL formulated with one or more radioprotectants and may include a buffer. The pH range of the solution is preferably 5.0 to 7.0. As discussed, radioprotectants or stabilizers may be one or more ascorbate compounds and optionally together with one or more gentisate compounds. The ¹⁷⁷Lu-PSMA I&T also may be provided in a multi-dose format or packaging, such as a multi-dose vial that contains multiple doses, such as 2, 3, 4, 5, 6, 7, 8, 9, or 10 or more doses of ¹⁷⁷Lu-PSMA I&T.

Preferred ¹⁷⁷Lu-PSMA I&T compositions for administration to a subject are also disclosed in U.S. Pat. Nos. 11,129,921 and 11,491,246.

### Methods of Treatment

As discussed, ¹⁷⁷Lu-PSMA I&T (including structures 2A and/or 2B above) is used to treat cancers, including a PSMA expressing cancer.

As discussed, in one aspect, methods are provided for treating a subject suffering from prostate cancer, comprising: a) identifying a subject exhibiting prostate cancer progression during or after treatment with one or more androgen receptor inhibitor agents, and wherein subject is chemotherapy-naïve before treatment with one or more androgen receptor inhibitor agents; b) administering to the identified subject an amount of a lutetium-177 labeled PSMA-targeted radioligand agent providing from a 4 to 10 Gbq dose, wherein the subject is dosed at least four times, at four week or longer intervals between doses, with the lutetium-177 labeled PSMA-targeted radioligand agent. In preferred aspects, the lutetium-177 labeled PSMA-targeted radioligand agent is a complex of lutetium-177 and EuK-Sub-kf-iodo-y-DOTAGA.

In particular aspects, ¹⁷⁷Lu-PSMA I&T (including structures 2A and/or 2B above) is used to treat prostate cancer, for example non-metastatic prostate cancer and metastatic prostate cancer, including hormone sensitive prostate cancer, castration resistant prostate cancer (CRPC) and drug-resistant prostate cancer, particularly an anti-androgen drug (e.g., abitaterone, enzalutamide, apalutamide and/or darolutamide) resistant prostate cancer.

In various methods, ¹⁷⁷Lu-PSMA I&T (including structures 2A and/or 2B above) can be administered to a subject such as a human in an amount effective to treat the cancer (e.g., reduction of tumor size), such as at a dose of about 0.1 GBq to about 30 GBq be suitably administered from a unit dose in a vial or a syringe or as a bulk solution in a vial or a syringe prepared from a cold-kit prepared with lutuetium-177 at a local or central nuclear pharmacy or through cGMP central manufacturing.

In certain embodiments, the subject is suffering from prostate cancer such as one or more of castration-sensitive prostate cancer, castration-resistant prostate cancer, metastatic castration-resistant prostate cancer, advanced stage prostate cancer, drug-resistant prostate cancer such as anti-androgen-resistant prostate cancer (e.g., enzalutamide-resistant prostate cancer, abiraterone-resistant prostate cancer, bicalutamide-resistant prostate cancer), docetaxel-resistant prostate cancer, PARP resistant prostate cancer, radium chloride resistant prostate cancer, AR-V7-induced drug-resistant prostate cancer such as AR-V7-induced enzalutamide-resistant prostate cancer, AKR1C3-induced drug-resistant prostate cancer such as AKR1C3-induced enzalutamide-resistant prostate cancer, and combinations thereof.

In particular embodiments, the subject is a human suffering metastatic castration-resistant prostate cancer and an effective amount of ¹⁷⁷Lu-PSMA I&T (including ¹⁷⁷Lu-PSMA I&T having an optical excess of structure 2A) is administered to the subject to treat the prostate cancer.

In additional particular embodiments, the subject is a human suffering oligometastatic hormone sensitive prostate cancer and an effective amount of ¹⁷⁷Lu-PSMA I&T (including ¹⁷⁷Lu-PSMA I&T having an optical excess of structure 2A) is administered to the subject to treat the prostate cancer.

In further particular embodiments, the subject is a human suffering metastatic castration-resistant prostate cancer and an effective amount of ¹⁷⁷Lu-PSMA I&T including ¹⁷⁷Lu-PSMA I&T having an optical excess of structure 2A) is administered to the subject to treat the prostate cancer.

The effective amount of ¹⁷⁷Lu-PSMA I&T (including ¹⁷⁷Lu-PSMA I&T having an optical excess of structure 2A) radiopharmaceutical administered to a patient will generally be determined by considering the patient record. However, the effective amount suitably may be within a range of about 0.1 GBq to 30 GBq per dose. More specifically, the dose may range from about 1 GBq to about 20 GBq or about 30 GBq per dose subject, for example, about 1, 1.5, 2, 2.5, 3, 3.5, 4, 4.5, 5, 5.5, 6, 6.5, 6.8, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21.5, 22, 22.5, 23, 23.5, 24, 24.5, 25, 25.5, 26, 26.5, 27, 27.5, 28, 28.5, 29, 29.5 or 30 GBq per dose of ¹⁷⁷Lu-PSMA I&T (including ¹⁷⁷Lu-PSMA I&T having an optical excess of structure 2A), or any range between two of the above values. The dose can be administered from a unit dose in a vial or a syringe or as a bulk solution in a vial or a syringe prepared from a cold-kit prepared with lutetium-177 at a local or central nuclear pharmacy or through cGMP central manufacturing.

If necessary or desirable, the treatment may involve more than one administration of an effective amount of ¹⁷⁷Lu-PSMA I&T (including ¹⁷⁷Lu-PSMA I&T having an optical excess of structure 2A). It is generally beneficial to repeat the administration of ¹⁷⁷Lu-PSMA I&T (including ¹⁷⁷Lu-PSMA I&T having an optical excess of structure 2A) to the subject after 7 to 56 days, such as at a 4 to 8 week interval.

In a particularly preferred protocol, the ¹⁷⁷Lu-PSMA I&T (including ¹⁷⁷Lu-PSMA I&T having an optical excess of structure 2A) dosage form is a sterile aqueous solution that is administered by intravenous injection. The dosing regimen may include multiple infusions such as 1, 2, 3, 4, 5, 6, 7, 8, 9 or 10 infusions at effective dosages such as of 6.8 GBq+/−10% each, administered about 1, 2, 3, 4, 5, 6, 7, 8, 9 or 10 weeks apart.

### Combination Therapy

¹⁷⁷Lu-PSMA I&T (including structures 2A and/or 2B above) suitably may be administered to a subject in conjunction or combination with one or more other therapeutic agents, particularly one or more other chemotherapeutic agents.

In one aspect, a subject may receive treatment with ¹⁷⁷Lu-PSMA I&T (including structures 2A and/or 2 above) in combination with a regime of docetaxel and/or prednisone, particularly for a subject suffering from castration resistant prostate cancer.

In another aspect, a subject may receive treatment with ¹⁷⁷Lu-PSMA I&T (including structures 2A and/or 2B above) in combination with a regime that can include chemotherapy such as docetaxel; cisplatin; gemcitabine; cisplatin/gemcitabine; cabazitaxel; one or more antiandrogens such as one or more LHRH agonists, such as leuprolide and goserelin, or antagonists (e.g. firmagon and relugolyx); one or more antiandrogens such as flutamide, nilutamide, bicalutamide, cyproterone, abiraterone, enzalutamide, darolutamide and apalutamide; one or more PARP inhibitors such as olaparib, rucaparib or niraparib, particularly for a subject suffering from prostate cancer including metastatic castration resistant prostate cancer.

In additional aspects, a subject may receive treatment with ¹⁷⁷Lu-PSMA I&T (including structures 2A and/or 2B above) in combination with an immunotherapy regime which may include adoptive cell therapies or adoptive immunotherapy.

For example, to treat a patient suffering from cancer, ¹⁷⁷Lu-PSMA I&T (including structures 2A and/or 2 above) may be administered in combination with immune effector cells (e.g., T cells, NK cells) engineered to express a Chimeric Antigen Receptor (e.g. CAR T-cell therapy), including to treat a cancer or a disease associated with expression of a tumor antigen.

For a patient suffering from cancer including prostate cancer, ¹⁷⁷Lu-PSMA I&T (including structures 2A and/or 2B above) also may be administered in combination with other immune-based therapies such as sipuleucel-T (Provenge) or other immune-boosting approaches including antibody treatments. For instance, in one protocol, ¹⁷⁷Lu-PSMA I&T (including structures 2A and/or 2B above) may be administered in combination with one or more monoclonal antibodies such as pembrolizumab (Keytruda), ipilimumab (Yervoy) and/or nivolumab (Opdivo) for treating a patient suffering from cancer, particularly prostate cancer.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need of treatment as disclosed herein. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

### Packaged ¹⁷⁷Lu-PSMA I&T and Kits

Treatment kits containing ¹⁷⁷Lu-PSMA I&T may be employed, including cold kits where the ¹⁷⁷Lu-PSMA I&T (including structures 2A and/or 2B above) can be prepared shortly before administration such as in a medical facility, for example a hospital laboratory or nuclear pharmacy. In such a kit, EuK-Sub-kf-iodo-y-DOTAGA may be provided in a vial or other container in lyophilized or other form separate from lutetium-177. The EuK-Sub-kf-iodo-y-DOTAGA and lutetium-177 are reacted as disclosed herein at the medical facility to provide ¹⁷⁷Lu-PSMA I&T (including 2A and/or 2B above) which then can be promptly administered to a patient.

In a further aspect, packaged preparations or products of ¹⁷⁷Lu-PSMA I&T (including structures 2A and/or 2B above) also may be utilized. A packaged preparation may comprise 1) ¹⁷⁷Lu-PSMA I&T and optionally 2) instructions for using ¹⁷⁷Lu-PSMA I&T for treating a cancer such as prostate cancer. Preferably, the packaged preparation will comprise a therapeutically effective amount of ¹⁷⁷Lu-PSMA I&T (including structures 2A and/or 2B above).

In certain exemplary packaged preparations or products, ¹⁷⁷Lu-PSMA I&T (including structures 2A and/or 2B above) suitably can be packaged in suitable containers labeled, for example, for use as a therapy to treat a subject suffering from prostate cancer. The containers can include ¹⁷⁷Lu-PSMA I&T and suitably one or more ascorbate compounds and one or more gentisate compounds as disclosed herein. A product can include a container (e.g., a vial or the like) containing ¹⁷⁷Lu-PSMA I&T. In addition, an article of manufacture or kit further may include, for example, packaging materials, instructions for use, syringes, delivery devices, for treating the targeted condition, such as prostate cancer or other cancer.

A packaged system or product may also include a legend (e.g., a printed label or insert or other medium (e.g., an audio or video file) describing the product's use). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents.

As discussed above, preferred kits may suitably comprise a) a pharmaceutical preparation of lutetium-177 labeled PSMA-targeted radioligand agent suitable for intravenous administration to a patient, and in an amount sufficient to provide from a 4 to 10 Gbq dose; and b) a package insert containing instructions for use of the agent in the treatment of a subject exhibiting prostate-specific membrane antigen (PSMA)-positive metastatic castration-resistant prostate cancer (mCRPC) who have previously been treated with androgen receptor (AR) pathway inhibition but which patient is taxane chemotherapy-naïve or taxane chemotherapy adverse (i.e., ineligible or averse to chemotherapeutic treatment options).

In certain aspects, suitably, the instructions of the package insert include guidance for dosing the patient with the lutetium-177 labeled PSMA-targeted radioligand therapy at least four times and at four week or longer intervals between doses.

In certain aspects, the package insert may suitably further include an indication that the shelf life for the lutetium-177 labeled PSMA-targeted radioligand is 120 hours from end of synthesis and formulation of the pharmaceutical preparation of the lutetium-177 labeled PSMA-targeted radioligand for use in the kit.

In certain aspects, the package insert suitably further includes instructions for selecting patients using a PSMA-targeted imaging agent.

In certain aspects, the package insert suitably further includes instructions for ensuring patients increase oral fluid intake and advise patients to void including as often as possible to reduce bladder radiation.

In certain aspects, in a kit, the lutetium-177 labeled PSMA-targeted radioligand agent is a complex of lutetium-177 and EuK-Sub-kf-iodo-y-DOTAGA.

In certain aspects, in a kit, lutetium-177 EuK-Sub-kf-iodo-y-DOTAGA complex is provided in a single dose form sufficient to provide 6.8 GBq+/−10% within shelf-life.

In certain aspects, the package insert further includes instructions for administration of the lutetium-177 EuK-Sub-kf-iodo-y-DOTAGA complex up to four times a patient with 8-week intervals between each dose.

In certain aspects, a pharmaceutical preparation includes Sodium Ascorbate a concentration of 55-75 mg/mL and Sodium Gentisate at a concentration of 16-36 mg/mL, and has a pH in the range of 5.0-7.0.

In certain aspects, the pharmaceutical preparation has a radioactive concentration (RAC) of 0.5 to 1.2 GBq/mL, more preferably 0.79-0.86 GBq/mL±10%, at end of synthesis and formulation of the pharmaceutical preparation.

In certain aspects, the pharmaceutical preparation has a total peptide content of less than or equal to 250 μg (sum of EuK-Sub-kf-iodo-y-DOTAGA and lutetium-177 EuK-Sub-kf-iodo-y-DOTAGA complex), preferably a total peptide content in the range of 180-250 kg.

In certain aspects, the pharmaceutical preparation is provided in a single dosage vial, and the kit optionally further comprises a disposable syringe with syringe shield for example to facilitate handheld infusion administration.

In certain aspects, the pharmaceutical preparation is provided in a disposable syringe with syringe shield, for example to facilitate handheld infusion administration.

In certain aspects, a kit may include a radiation shielded pig (such as lead or tungsten pig) in which the pharmaceutical preparation is transported in the packaged kit.

In certain aspects, a package insert may further include instructions for patient eligibility, including that patients must have progressive mCRPC at the time of consent based on one or more or all of the following criteria:
a) Serum/plasma PSA progression defined as increase in PSA greater than 25% and >2 ng/mL above nadir;
b) Progression on previous treatment with one ARAT in either the CSPC or CRPC setting;
c) PSMA-PET scan (i.e., 68Ga-PSMA-11 or 18F-DCFPyL) positive;
d) Castrate circulating testosterone levels (<1.7 nmol/L or <50 ng/dL); and e) Adequate bone marrow, renal and liver organ function.

The following non-limiting examples are illustrative.

### Example 1

Aqueous compositions of ¹⁷⁷Lu-PSMA-I&T are prepared as set forth in Example 1 of U.S. Pat. No. 11,129,912.

### Example 2: Clinical Use

A primary efficacy objective is to determine the efficacy of ¹⁷⁷Lu-PSMA I&T versus abiraterone or enzalutamide is evaluated in delaying radiographic progression in patients with mCRPC who have cancer progression progressed on androgen receptor-axis-targeted therapies (ARAT).

A primary endpoint of the study may include radiological progression-free survival (rPFS) assessed by Blinded Independent Central Review (BICR) using Response Evaluation Criteria in Solid Tumors (RECIST).

Additional endpoints may include proportion of patients with partial or complete response (PR or CR, respectively) by BICR based on RECIST 1.1 criteria (soft tissue) and PCWG3 criteria (bone).

Two treatment groups (Arm A and Arm B) of human patients are selected. Arm B treatment includes enzalutamide (160 mg orally qd) or abiraterone acetate (abiraterone) (1000 mg orally qd with: 5 mg bid prednisone or 0.5 mg qd dexamethasone). Patients randomized to Arm B will receive all the benefits of standard clinical care for mCRPC. Following progression on control arm treatment, patients will be assessed and, if eligible, will crossover to ¹⁷⁷Lu-PSMA I&T treatment.

### Study Design

The study evaluates the efficacy and safety of ¹⁷⁷Lu-PSMA I&T in patients with mCRPC who have progressed on ARAT therapy.

The study may include three phases: Dosimetry, Randomized Treatment, and Long-term Follow-up.

The study may commence with a 25-patient safety and dosimetry lead-in and proceed to a randomization phase in approximately 390 patients at the same dose and regimen if pre-specified safety criteria are met. All patients will be followed in long-term follow-up for at least 5 years from the first therapeutic dose, death, or loss to follow-up. Patient groups size may vary.

In one preferred protocol, all patients will undergo screening such as within 6 weeks before enrollment or randomization to assess eligibility and must be chemo-naïve for CRPC, unfit or unwilling to receive chemotherapy, have documented progression on ARAT therapy (e.g., abiraterone, enzalutamide, apalutamide, darolutamide), and willing to undergo treatment with second-line ARAT therapy. Once all screening assessments have been conducted, patients will undergo PSMA imaging with ¹⁸FDCFPyL or ⁶⁸Ga-PSMA-11 PET/CT to confirm PSMA expression eligibility.

Patients that meet eligibility in the Dosimetry Phase will be administered ¹⁷⁷Lu-PSMA I&T, for example at a dose of 6.8 GBq (±10%) every 8 weeks for 4 cycles. Concurrent with each patient's first treatment cycle, whole body planar acquisitions will be collected at 5 imaging times: 0.5-2 h (pre-void), 24 h (±4 h), 48 h (±4 h), 72 h (±4 h) and 140-196 h. SPECT will also be encouraged, but not required, at 24 h or 48 h time points.

Once dosimetry and safety data are generated to confirm the selected dose meets pre-specified criteria and the DSMB has provided an approval to proceed, the Randomized Treatment Phase will commence. The randomized treatment phase will open to US sites after all patients in the dosimetry phase have completed the treatment follow-up period (i.e. 8 weeks after last dose). Patients that meet all eligibility will be randomized in a 2:1 ratio to receive either:
Arm A (n=260): ¹⁷⁷Lu-PSMA I&T 6.8 GBq (±10%) every 8 weeks for 4 cycles, or
Arm B (n=130): enzalutamide (160 mg orally qd) or abiraterone (1000 mg orally qd with: 5 mg bid prednisone or 0.5 mg qd dexamethasone).

The primary objective of the study is to determine the efficacy of ¹⁷⁷Lu-PSMA I&T versus abiraterone or enzalutamide in delaying radiographic progression. Secondary objectives include overall response, overall survival, effect on PSA kinetics and safety and tolerability of ¹⁷⁷Lu-PSMA I&T compared with the control arm. Exploratory objectives are to evaluate the effect on cancer-related pain, impact on HRQoL, and efficacy of PSMA-PET for patient selection with ¹⁷⁷Lu-PSMA I&T therapy.

Patients in the Dosimetry Phase and Arm A will be assessed every 2 weeks and patients in Arm B will be assessed every 4 weeks until completion of the treatment phase (EOT; Week 32). In both the Dosimetry and Randomized Treatment Phases, a patient is considered to have completed the study if he has completed the treatment phase at study Week 32 (EOT Visit). If patients have not progressed by EOT, disease assessments based on CT/MRI and whole-body bone scan will continue every 8 weeks until radiologic progression by BICR.

Patients randomized to Arm B who experience radiographic progression per BICR, have not started an intervening treatment and have no uncontrolled AEs will be eligible to cross over to receive 6.8 GBq (±10%) of ¹⁷⁷Lu-PSMA I&T every 8 weeks for 4 cycles. Patients that switch will be followed for safety and efficacy based on the SOA with the exception of ECGs, correlative blood draws and BICR assessed imaging interpretation.

A subset of up to 20 patients in Arm A from selected sites will have PK assessments with blood and urine samples.

Efficacy will be assessed by CT/MRI scans of the chest, abdomen, and pelvis, whole body bone scans, PSA measurements, Eastern Cooperative Oncology Group (ECOG), a health-related QoL (HRQoL) questionnaire, pain questionnaires, and skeletal events review.

Safety will be assessed by measurement of weight, physical examinations, vital signs, ECGs, blood chemistry and hematologic parameters, review of AEs/serious AEs (SAEs), review of concomitant medications.

Patients on the control arm (Arm B) will be assessed based on the same procedures as the investigational arm (Arm A) except for ECGs performed post baseline and PK samples drawn in Arm A only and treatment compliance checked at each visit for Arm B.

An independent Data Safety Monitoring Committee (iDSMB) will monitor ongoing safety data (AEs and laboratory test results).

The Long-Term Follow-up Phase consists of a planned clinic visit or phone call every 3 months to assess survival status, late-radiation related toxicities, and new anti-cancer therapies for at least 5 years from the first therapeutic dose, death, or loss to follow up.

### Study Population

Twenty-five patients are enrolled in the Dosimetry Phase of the study. A total of 390 patients will be randomized in a 2:1 ratio in the Treatment Phase: 260 patients will receive ¹⁷⁷Lu-PSMA I&T and 130 patients will receive abiraterone or enzalutamide.

Prospective approval of protocol deviations to recruitment and enrollment criteria, also known as protocol waivers or exemptions, is not permitted.

### Inclusion Criteria

Patients are eligible to be included in the study only if all of the following criteria apply:
1. Male aged 18 years or older.
2. Histological, pathological, and/or cytological confirmation of adenocarcinoma of the prostate.
3. Ineligible or averse to chemotherapeutic treatment options.
4. Patients must have progressive mCRPC at the time of consent based on at least 1 of the following criteria:
a. Serum/plasma PSA progression defined as increase in PSA greater than 25% and >2 ng/mL above nadir, confirmed by progression at 2 time points at least 3 weeks apart.
b. Soft-tissue progression defined as an increase≥20% in the sum of the diameter (SOD) (short axis for nodal lesions and long axis for non-nodal lesions) of all target lesions based on the smallest SOD since treatment started or the appearance of one or a new lesion.
c. Progression of bone disease: evaluable disease or one new bone lesion by bone scan
5. Progression on previous treatment with one ARAT (abiraterone or enzalutamide or darolutamide or apalutamide) in either the CSPC or CRPC setting.
6. PSMA-PET scan (i.e., 68Ga-PSMA-11 or 18F-DCFPyL) positive as determined by the sponsor's central reader.
7. Castrate circulating testosterone levels (<1.7 nmol/L or <50 ng/dL).
8. Adequate organ function, independent of transfusion:
a. Bone marrow reserve:
i. White blood cell (WBC) count≥2.5×109/L OR absolute neutrophil count (ANC)≥1.5×109/L.
ii. Platelets≥100×109/L.
iii. Hemoglobin≥8 g/dL.
b. Liver function:
i. Total bilirubin≤1.5×institutional upper limit of normal (ULN). For patients with known Gilbert's syndrome, ≤3×ULN is permitted.
ii. ALT or AST≤3.0×ULN.
c. Renal function:
i. Serum/plasma creatinine≤1.5×ULN or creatinine clearance≥50 mL/min based on Cockcroft-Gault formula.
d. Albumin≥30 g/L.
9. Human immunodeficiency virus-infected patients who are healthy and have a low risk of acquired immunodeficiency syndrome-related outcomes are included in this trial.
10. For patients who have partners of childbearing potential: Partner and/or patient must use a method of birth control with adequate barrier protection, deemed acceptable by the principal investigator during the study and for 6 months after last study drug administration.
11. Willing to initiate ARAT therapy (either enzalutamide or abiraterone), pre-specified by investigator, if randomized to Treatment Arm B.
12. ECOG performance status 0 to 1.
13. Willing and able to comply with all study requirements and treatments (including ¹⁷⁷Lu-PSMA I&T) as well as the timing and nature of required assessments.
14. Signed informed consent.

### Exclusion Criteria

Patients are excluded from the study if any of the following criteria apply:
1. If noted in pathology report, prostate cancer with known significant (>10% present in cells) sarcomatoid or spindle cell or neuroendocrine components. Any small cell component in the cancer should result in exclusion.
2. Prior treatment for prostate cancer≤28 days prior to randomization, with the exclusion of firstline local external beam, ARAT, luteinizing hormone-releasing hormone (LHRH) agonist or antagonist therapy, or non-radioactive bone-targeted agents.
3. Any prior cytotoxic chemotherapy for CRPC (e.g., cabazitaxel or docetaxel); chemotherapy for hormone-sensitive prostate cancer (HSPC) is allowed if the last dose was administered>1 year prior to consent.
4. Prior treatment with systemic radionuclides (e.g. radium-223, rhenium-186, strontium-89).
5. Prior immuno-therapy, except for sipuleucel-T.
6. Prior PSMA-targeted radioligand therapy.
7. Prior poly ADP ribose polymerase (PARP) inhibitor for prostate cancer.
8. Patients who progressed on 2 or more lines of ARATs.
9. Patients receiving bone-targeted therapy (e.g. denosumab, zoledronic acid) must be on stable doses for at least 4 weeks prior to randomization.
10. Administration of an investigational agent≤60 days or 5 half-lives, whichever is shorter, prior to randomization.
11. Major surgery≤30 days prior to randomization.
12. Estimated life expectancy<6 months as assessed by the principal investigator.
13. Presence of liver metastases>1 cm on abdominal imaging.
14. A superscan on bone scan defined as a bone scan that demonstrates markedly increased skeletal radioisotope uptake relative to soft tissues in association with absent or faint genitourinary tract activity.
15. Use of opioids for cancer-related pain≤30 days prior to consent.
16. Known presence of central nervous system metastases.
17. Contraindications to the use of planned ARAT therapy.
18. Active malignancy other than low-grade non-muscle-invasive bladder cancer and non-melanoma skin cancer.
19. Concurrent illness that may jeopardize the patient's ability to undergo study procedures.
20. Serious psychological, familial, sociological, or geographical condition that might hamper compliance with the study protocol and follow-up schedule. Patients that travel need to be capable of repeated visits even if they are on the control arm.
21. Symptomatic cord compression, or clinical or radiologic findings indicative of impending cord compression.
22. Concurrent serious (as determined by the investigator) medical conditions, including, but not limited to, New York Heart Association class III or IV congestive heart failure, unstable ischemia, uncontrolled symptomatic arrhythmia, history of congenital prolonged QT syndrome, uncontrolled infection, known active hepatitis B or C, or other significant co-morbid conditions that in the opinion of the investigator would impair study participation or cooperation.

### Screening

Patients will be screened within 6 weeks before randomization.

### Study Intervention

The objective of the Dosimetry Phase is to evaluate dosimetry in all of the standard organs, including the kidneys, salivary glands, and lacrimal glands. Individual dosimetry estimates and summary statistics will be generated by a central dosimetry core laboratory.

Prior to entry into the Dosimetry Phase, patients will sign an ICF and undergo screening procedures including a PSMA-PET scan. A total of 25 patients that meet all eligibility requirements will be administered 6.8 GBq (±10%) of 177Lu-PSMA I&T every 8 weeks for 4 cycles.

A baseline CT scan will be collected for the purpose of determination of kidney volumes for patient-specific correction in the dosimetry analysis process.

Concurrent with each patient's first treatment cycle, 5 whole body conjugate-view planar images will be collected at the times listed below on a dual-headed gamma camera capable of whole-body scanning with a medium energy collimator. Images should include the entire body from vertex to feet, and all parts of the body including the arms should be contained completely in the field of view. An imaging standard with a known amount of activity is required to be present in each image collected. The data and time of each image collection, and the scan speed of each image will be recorded. Although not required, sites also will be encouraged to collect quantitative or semiquantitative SPECT images at either the 24 or 48 h time points below for supplemental dosimetry analyses. If only a single optional SPECT image is to be collected, 48 hours is a preferred time of collection, followed by 24 hours. Further details of the whole body planar acquisitions and SPECT acquisitions will be contained in the imaging manuals.

### Whole Body Image Acquisition Times:

0.5-2 h (pre-void)
24 h (±4 h)
48 h (±4 h)
72 h (±4 h)
140-196 h

A central dosimetry core laboratory will perform image quantification, kinetic modeling, and dosimetry analysis across all standard organs utilizing the standard MIRD/RADAR methodology.

Regions of interest (ROIs) will be constructed on the images for all organs/tissues that show uptake, and the activity in the ROIs will be quantified at each time point. Red marrow activity will be estimated using marrow ROIs or a blood-based methodology. Kinetic modeling will be performed to obtain normalized number of disintegrations, and the OLINDA/EXM v2 software will be utilized to produce dosimetry estimates based on the generated activity-time curves and derived residence times for all organs/tissues. Organ masses derived from CT will be used to more accurately estimate the kidney absorbed dose.

After each of the 4 treatment cycles, patients will be assessed every 2 weeks until Week 32 (8 weeks after the last cycle) to monitor tolerability and safety (i.e., severity, frequency and duration of AEs/SAEs, vital signs, ECG, hematology and chemistry). A safety evaluation of all AEs including xerostomia, dry eyes, and kidney function, and lab abnormalities will be followed closely for worsening in severity or resolution and to evaluate if dose modifications for subsequent cycles may be warranted.

An iDSMB will be convened to evaluate safety results after the first 5 patients have completed treatment, or sooner, if requested by the Medical Monitor. The iDSMB will evaluate safety until all 25 patients are enrolled and complete treatment.

Efficacy will also be assessed based on radiographic tumor assessments, PSA blood samples, ECOG and QoL. Radiographic response will be collected and assessed by BICR throughout the Dosimetry Phase.

### Pre-Specified Criteria for Dose Selection on the Randomized Phase

The following criteria will be used to confirm the dose will remain the same for the Randomized Treatment Phase:
Dosimetry data obtained from the dosimetry and safety lead-in demonstrated a mean absorbed renal dose≤20 gray (Gy).
No clinical toxicity concerns in the dosimetry and safety lead-in were identified by the independent Data Safety Monitoring Board (iDSMB), the Medical Monitor, the Sponsor or FDA based on ongoing data review and Stopping Rules.
The Sponsor does not wish to increase the dose or shorten the intervals between doses due to preliminary efficacy observed based on PSA response.
   The following criteria will be used to determine a dose modification in the Randomized Treatment
If a mean absorbed renal dose obtained from the dosimetry and safety lead-in is >20 Gy, the fourth dose for each patient's treatment regimen in the Randomized Treatment Phase will be reduced to a level calculated from the linear dosimetry data curve to ensure a mean absorbed renal dose of ≤20 Gy.
If >45% of patients in the dosimetry and safety lead-in experience a dose reduction to 5 GBq after their first therapeutic dose based on the Dose Modification criteria set forth below, a reduced dose of 5 GBq will be implemented for all cycles in the Randomized Treatment Phase.
If preliminary efficacy is not observed based on a descriptive review of PSA reduction, the Sponsor will stop the study and meet with FDA before implementing a dose escalation paradigm.

Randomized Treatment Phase Once dosimetry and safety data are generated to confirm the selected doses meets pre-specified criteria and the DSMB has provided an approval to proceed, the Randomized Treatment Phase will commence. The randomized treatment phase will open to sites after all patients in the dosimetry phase have completed the entire follow-up period (i.e. 8 weeks after last doe) or earlier if FDA agreement is obtained. Patients will sign an ICF and undergo screening procedures including a PSMA-PET scan. Following determination of eligibility, patients will be randomized 2:1 in the following groups:
Arm A, in which approximately 260 patients will receive ¹⁷⁷Lu-PSMA I&T (6.8 GBq (±10%) every 8 weeks for 4 cycles).
Arm B, in which approximately 130 patients will receive enzalutamide (160 mg orally qd) or abiraterone (1000 mg orally qd with: 5 mg bid prednisone or 0.5 mg qd dexamethasone).

Randomization will be stratified by prior taxane treatment for castration-sensitive prostate cancer (CSPC) (yes or no), prior use of bisphosphonates (yes or no), metastatic status on prior ARAT (M0 or M1), and measurable disease at study entry (yes or no).

Patients in both Treatment Arms will be highly encouraged to remain on their randomized treatment until objective radiographic disease progression as assessed by BICR. All patients will undergo disease assessments by whole body bone scans and CT/MRI imaging of the chest, abdomen, and pelvis. Patients will also be evaluated for changes in Eastern Cooperative Oncology Group (ECOG), PSA measurements by a central laboratory, HRQoL questionnaire, pain questionnaires, and skeletal events review. Safety will be assessed by measurement of weight, physical examinations, vital signs, electrocardiograms (ECGs), blood chemistry and hematologic parameters, review of AEs/SAEs and review of concomitant medications.

ARAT administration in the control arm will be modified according to the approved product labeling. The dose of 177Lu-PSMA I&T can be modified as per the guidelines set forth below.

Patients are suitably assessed at the clinic every 2 weeks (±2 days) in Arm A and every 4 weeks (±2 days) in Arm B until completion of the treatment phase (EOT; 8 weeks after last dose). In the absence of BICR-assessed radiographic progression by EOT, patients will continue to be followed every 8 weeks until radiographic progression by BICR and follow all procedures indicated within the SOA for these visits. Every effort should be made to keep patients on assigned treatment until BICR-assessed radiographic progression. If the investigator elects to initiate alternative anti-cancer therapy before BICR-assessed radiological progression. Discontinuation of Study Intervention, and patients will continue to be followed every 8 weeks until BICR-assessed radiographic progression, irrespective of initiation of subsequent anti-cancer therapy. Once a patient has demonstrated BICR-assessed radiographic progression, they will complete the Progression Visit and:
if EOT/Safety follow-up has already occurred (and patient does not crossover if randomized to Arm B), they will enter the Long-Term Follow-up (LTFU) Phase for at least 5 years from first therapeutic dose, death, or loss to follow-up.
if EOT/Safety follow-up has not occurred (and patient does not crossover if randomized to Arm B), they will remain on their study schedule until 8 weeks after last dose, complete the EOT/Safety Follow-Up Visit, and then enter LTFU.
Arm B patients who cross over will proceed to Cycle 1, Week 0, and follow the Arm A SOA (except ECGs and correlative samples)

If the primary rPFS analysis occurs before patients progress by BICR, patients will continue their original study schedule until investigator-assessed radiographic progression, death or loss to follow-up.

An iDSMB will monitor ongoing safety data (AEs/SAEs, ECGs, laboratory test results) on a quarterly basis throughout the Treatment Phase. A subset of up to 20 patients in Arm A will also be evaluated for PK.

Patients in Arm B who experience radiographic progression per BICR, have not started an intervening treatment, and have no uncontrolled AEs will be eligible for consent to crossover to receive 6.8 GBq (±10%) of ¹⁷⁷Lu-PSMA I&T every 8 weeks for 4 cycles. Radiologic assessment of second progression will be performed by the investigator and patients will be monitored for safety based on the schedule set forth below.

### Long-Term Follow-Up Phase

The Long-Term Follow-up Phase for the Dosimetry and Randomization Treatment Phases consists of a planned clinic visit or phone call every 3 months to assess survival status, late-radiation related toxicities, second progression, and new anti-cancer therapies for at least 5 years, death, or loss to follow up. The overall study ends when the last subject completes the last study related phone-call or visit, discontinues from the trial or is lost to follow-up.

⁶⁸Ga-PSMA-11 and ¹⁸F-DCFPyL

The ⁶⁸Ga-PSMA-11 radiopharmaceutical will be administered intravenously at a dose of 5 mCi+/−10%. The ¹⁸F-DCFPyL radiopharmaceutical will be administered intravenously at a dose of 9 mCi+/−10%.

### ¹⁷⁷Lu-PSMA I&T Storage and Handling

To limit exposure, ¹⁷⁷Lu-PSMA I&T is stored inside a lead-shield container. It is shipped in an International Air Transportation Association (IATA) approved package and marked with an appropriate radioactive label. Upon receipt, ¹⁷⁷Lu-PSMA I&T can remain within the shipping container until needed for injection. Shipping documents should include the ART.

The drug is stored at room temperature (15-30° C.) in a lead pot at the clinical site. The shelf life is 2 days post-activity reference time when stored in its original packaging, after which the drug product should be appropriately discarded.

### ¹⁷⁷Lu-PSMA I&T Preparation and Administration

Administration of ¹⁷⁷Lu-PSMA I&T must occur by the labeled expiration time. The time of all measurements should be documented in reference to a single clock. If possible, the clock on the dose calibrator should be synchronized to this reference clock. The amount of radioactivity in the vial should be measured before and after each administration. The decay-corrected administered dose will be calculated in the eCRF once the dose measurements and times are entered in the eCRF.

Before ¹⁷⁷Lu-PSMA I&T administration, an indwelling IV catheter should be inserted into the antecubital vein (preferably). The IV line should be flushed with ≥10 mL of normal saline to confirm patency. The dose should be administered via slow push or with an automatic pump over a period of 1 to 10 minutes with careful observation to ensure the dose is not extravasated. Upon completion of the administration, the line should be flushed with ≥10 mL of normal saline.

If necessary, the infusion may be interrupted to accommodate the patient (e.g., the patient needs to use the toilet, experiences nausea or vomiting, anxiety). The infusion should be continued unless the investigator determines that the administration represents an unacceptable risk, undue safety hazard, or if the patient's medical condition deteriorates rapidly and the infusion cannot continue or would interfere with medical or surgical care. If the infusion can be completed on the scheduled day, it should proceed even with interruptions.

### Control Arm

Patients randomized to Arm B will receive abiraterone or enzalutamide as defined below:
Patients progressing on either enzalutamide, darolutamide or apalutamide will receive abiraterone according to the approved product labeling of 1000 mg qd plus prednisone at a dose of 5 mg bid.
Patients progressing on abiraterone, will receive either: 1) Enzalutamide prescribed according to the approved product labeling of 160 mg qd or 2) Abiraterone prescribed according to the approved product labeling of 1000 mg qd plus dexamethasone at a dose of 0.5 mg qd.

Patient's treatment must remain the same throughout the Randomized Treatment Phase with the exception of dose modifications in accordance with product labeling.

### Treatment Compliance

¹⁷⁷Lu-PSMA I&T, ¹⁸F-DCFPyL, and ⁶⁸Ga-PSMA-11 are administered under the supervision of the investigator or qualified designee. Details of the study drug injection will be captured in each patient's eCRF.

The abiraterone or enzalutamide treatment regimen is self-administered as per standard clinical dosing. Patients will be instructed to take the treatment as per standard of care and appropriate competent authority approved drug labeling. Compliance with the prescribed dosing schedule will be assessed at each clinic visit and the information will be entered into the eCRF.

### Randomization, Stratification, and Blinding

Randomization is performed in a 2:1 ratio using IVRS/IWRS following confirmation of patient's eligibility and prior to initiating study treatment. The following steps are to be followed in the enrollment process:
The investigator obtains written informed consent from patients before any study—specific procedures are performed. A screening log of patients who were consented for the study should be kept by the investigator at the site.
All patients who signed consent are assigned a unique sequential enrollment number using the Interactive Voice/Web Response System (IVRS/IWRS). This enrollment number is to be used to identify the patient on study documents and the eCRF.
Upon determination of patient's eligibility, including PSMA avidity confirmation by BICR, randomization is performed using IVRS/IWRS. The patient should start study treatment (¹⁷⁷Lu-PSMA I&T or control arm) as soon as possible following randomization. The maximum length of time between randomization and the first dose of treatment for the control arm is 3 weeks. The timing for patients on 177Lu-PSMA I&T will be determined by the timelines for drug shipment and stability.

### Patient Stratification

Randomization will be stratified by prior taxane treatment for castration-sensitive prostate cancer (CSPC) (yes or no), prior use of bisphosphonates (yes or no), metastatic status on prior ARAT (M0 or M1), and measurable disease at study entry (yes or no).

### Prior and Concomitant Therapy

All treatments (medications, including over-the-counter or prescription medicines, vitamins, and/or herbal supplements, or medical procedures) that the patient completed within 30 days prior to randomization are to be recorded in the eCRF, including the reason for use; dates of administration including start and end dates; and dosage information including dose and frequency.

### Permitted/Prohibited Medications

### Permitted Medications

The following medications are allowed on both arms:
Patients without prior surgical castration must be taking and willing to continue taking LHRH analog treatment throughout the study.
Pre-specified continued use of glucocorticoids to prevent secondary mineralocorticoid excess syndrome is permitted but should not be added after randomization unless prescribed as part of the ARAT regimen for Arm B.
Pre-specified bisphosphonates or denosumab is permitted provided the dose is stable and started at least 4 weeks prior to study treatment but should not be started after randomization.
Palliative external beam radiation.
Palliative surgical procedures to treat skeletal-related events.
Opioids for cancer-related pain are not permitted≤30 days prior to consent, but may be initiated after randomization.

Investigator discretion should be used for any deviations from the guidelines above to ensure the patient's safety. Deviations should be recorded in the eCRF and communicated to the sponsor for evaluation of continuing eligibility.

### Prohibited Medications

In certain aspects, the following medications are prohibited during participation in the study:
Other investigational agents.
Other systemic radioisotopes.
Poly ADP ribose polymerase inhibitors.
Cytotoxic chemotherapy.
Hemi-body radiotherapy.

### Dose Modification

### Control Arm

Androgen receptor inhibitor (ARAT) administration in the control arm will be modified according to the approved product labeling.

### ¹⁷⁷Lu-PSMA I&T Dose Reduction

If a patient experiences any of the following toxicities, further dosing at 6.8 GBq should be suspended and subsequent doses should be reduced to 5 GBq once the subject meets criteria for resuming treatment:
Grade 3 or higher hematological toxicity, including: Severe neutropenia defined as ANC<1.0×109/L Thrombocytopenia defined as platelet count<50.0×109/L-25.0×109/L with ≥Grade 2 bleeding or Grade 4 thrombocytopenia (platelet count<25×109/L) lasting 1 week with or without bleeding, or platelet count<10,000/μL at any time point.
Grade 3 or higher treatment-related nephrotoxicity defined as: 1) Creatinine elevation>3.0×ULN or 2) Kidney disease defined as CrCl<15 mL/min/1.73 m2.
Grade 3 or higher xerostomia, sialadenitis, or dry eyes that has not reduced to CTCAE v5.0 Grade 1, or the baseline value, by the next treatment cycle.
Other significant treatment-related toxicities deemed by the investigator to be dose-limiting.

Once dosing has been suspended, subjects should have CBC and/or serum chemistry with creatinine as well as AE monitoring evaluated every other week until the dose limiting toxicity has recovered back to no greater than CTCAE v5.0 Grade 1 or the baseline value, before dosing may be resumed at 5 GBq. If by 16 weeks following the previous dose, the values have not recovered, permanent discontinuation from further dosing should occur and the subject will be asked to remain in the study to follow disease progression, safety and survival.

### ¹⁷⁷Lu-PSMA I&T Dose Delay

In addition to the criteria specified above, the ¹⁷⁷Lu-PSMA I&T dose may also be delayed for any other reason at the Investigator's discretion and in consultation with the Medical Monitor.

Permanent discontinuation should occur if a dose is delayed greater than 16 weeks.

### Discontinuation of Study Intervention

Patients may discontinue the study treatment for the following reasons:
Patient's discretion.
AE/SAE.
Significant non-compliance with the study protocol procedures and assessments, as determined by the investigator and/or sponsor.
Objective radiographic progression by BICR as defined by RECIST 1.1 and PCWG3.
Unequivocal clinical progression: 1) Initiation of chronic opioid use for new onset of prostate cancer pain suspected due to disease progression. Chronic use is defined as daily use for more than 7 consecutive days or more than 10 days within a 14-day period, or 2) Immediate need to initiate cytotoxic chemotherapy, or 3) Radiation or surgical therapy for complications of prostate cancer progression, excluding palliative radiotherapy (in treatment of pain at site of bone metastases present at baseline, unless indicative of disease progression), or 4) Deterioration in ECOG performance status to ≥3 due to prostate cancer.
Permanent discontinuation should occur if a dose is delayed for greater than 16 weeks. The reason for discontinuing the study treatment is to be recorded in the eCRF, along with all relevant details (date of discontinuation, relevant symptoms, and applicable treatments). Any discontinuation of study treatment should be communicated to the sponsor within 48 hours of the site investigator being made aware of the event.
   If a patient discontinues study treatment before BICR-assessed radiographic progression is declared, they should be followed for radiographic progression by BICR and OS as per the Schedule of Assessments.
   Late stage radiation toxicities (e.g., secondary malignancies and renal toxicity) will be collected through the LTFU.
   Withdrawal of Patient from the Study

A patient is considered to be withdrawn from the study if he has discontinued study treatment as well as the protocol-mandated assessments. A patient may be withdrawn from the study for the following reasons:
Incorrect enrollment (i.e., enrollment despite not meeting the eligibility criteria).
Patient's discretion (i.e., voluntary withdrawal).
Loss to follow-up.
Death.

For all patients who withdraw consent, the reason should be recorded in the eCRF. If the patient withdraws consent for disclosure of future information, the sponsor may retain and continue to use any data collected before such a withdrawal of consent.

If a patient withdraws from the study, he may request destruction of any samples taken and not tested, and the investigator must document this in the site study records.

### Early Study Termination

Stopping rules apply to both the Dosimetry and Randomized Treatment Phases. The study will be suspended and possibly stopped for any of the following reasons:
Death in any patient in which the cause of death is unexpected and assessed as at least possibly related to ¹⁷⁷Lu-PSMA I&T and deemed in the judgement of the Sponsor to contraindicate further dosing of study patients.
Any treatment-related event, in the judgment of the Medical Monitor, deemed serious enough to warrant immediate review by the iDSMB. This may include any treatment related symptomatic and/or irreversible treatment-related grade 4 hematologic toxicity, nephrotoxicity, lacrimal gland toxicity (e.g., dry eyes), or salivary gland toxicity (e.g., xerostomia).
Any other safety finding assessed as related to ¹⁷⁷Lu-PSMA I&T that, in the opinion of the DSMB and Sponsor, contraindicates further dosing of study patients.

### Screening Procedures

The following procedures and assessments will take place once the patient has provided written informed consent and prior to randomization onto the study. Procedures conducted as part of the patient's routine clinical management (e.g., blood count) and obtained before signing of the ICF may be utilized for screening or baseline purposes provided the procedures met the protocol-specified criteria and were performed within the time frame defined in the SOAs.

All screening procedures needed to ascertain eligibility for the study must be completed within 6 weeks prior to enrollment or randomization. All data will be recorded in the eCRF.

### Investigations Timing

Clinical assessment Medical history, complete physical examination, morphometric measurements (height, weight), vital signs, ECG, ECOG, recording of SAEs, recording of prior medications, recording of concomitant medications (All subjects) within 6 weeks before enrollment/randomization.

Treatment history Past treatment for prostate cancer, including pathology and/or histology Tumor marker PSA Hematology WBC count, 3-part differential, RBC count, hemoglobin, hematocrit, mean corpuscular volume, platelet count Blood chemistry Electrolytes (sodium, potassium, calcium, chloride, bicarbonate, phosphate, magnesium), total protein, serum creatinine, albumin, glucose (non-fasting), ALP, LDH, CK, total bilirubin, AST, ALT.

Additional blood test

### Testosterone

### Radiology PSMA-PET

### Optional ¹⁸F-FDG PET

Within 30 days before randomization after all other eligibility requirements have been completed.
Imaging CT/MRI chest, abdomen, pelvis, using RECIST v1.1 criteria as outlined in PCWG3 recommendations;
Whole body bone scan, Up to 6 weeks before enrollment/randomization.

### Investigations Timing

Clinical assessment prior to ¹⁷⁷Lu-PSMA I&T administration
Limited physical examination, body weight measurement, ECOG
Prior to each ¹⁷⁷Lu-PSMA I&T infusion and the last visit of each cycle (weeks 4, 8, 12, 16, 20, 24 and 28) during the treatment period; and at EOT
Clinical assessment pre- and post ¹⁷⁷Lu-PSMA I&T administration
ECG Pre- and post-infusion (within 4 hr. post-dose) of all treatments with ¹⁷⁷Lu-PSMA I&T and at EOT
Tumor marker PSA Week of first ¹⁷⁷Lu-PSMA I&T treatment, prior to infusion, and every 4 weeks during the treatment period, at the progression visit, at EOT, and if progression has not occurred by EOT, every 8 weeks until progression
Hematology WBC count, WBC differential (3-part), RBC count, hemoglobin, hematocrit, mean corpuscular volume, platelet count
Prior to ¹⁷⁷Lu-PSMA I&T infusions and every 2 weeks between infusions, at progression the visit and at EOT
Blood chemistry Electrolytes (sodium, potassium, calcium, chloride, bicarbonate, phosphate, magnesium), total protein, serum creatinine, albumin, glucose (non-fasting), ALP, LDH, CK, total bilirubin, AST, ALT
Vital signs Blood pressure, heart rate, temperature, respiratory rate
Dosimetry Whole body planar images At baseline (0.5-2 hr. [pre-void], 24 hr. [±4 hr.], 48 hr. [±4 hr.], 72 hr. [±4 hr.], and 140-196 hr.).
Imaging CT/MRI chest, abdomen, pelvis, whole body bone scan
Weeks 8, 16, 24, EOT, and every 8 weeks until progression
Toxicity AEs Every visit from first dose of Investigational Product (i.e. PSMA
Imaging Agent) through EOT SAEs Every visit from Screening through
EOT Late stage radiation toxicities If progression has not occurred by EOT, every 8 weeks until LTFU

### Concomitant Medications

Review of concomitant medications every visit through EOT
If progression has not occurred by EOT, the use of anti-cancer therapy (although prohibited) will be collected every 8 weeks until progression
Opioid use Review of opioid medications Every visit until initiation of opioid use
Quality-of-life FACT-P Baseline and Weeks 8, 16, 24, and EOT
Pain assessment BPI-SF, AQA, review of skeletal events Day 1 and the last visit of each cycle (weeks 4, 8, 12, 16, 20, 24 and 28) during the treatment period and at EOT
If progression has not occurred by EOT, AQA will be completed every 8 weeks until progression

### Imaging PSMA PET Time of Progression

### ¹⁷⁷Lu-PSMA I&T Randomized Treatment Period

Patients randomized to Arm A will undergo the following investigations during the cycles of ¹⁷⁷Lu-PSMA I&T treatment.

### Investigations Timing

Clinical assessment prior to ¹⁷⁷Lu-PSMA I&T administration
Limited physical examination, body weight measurement, ECOG Prior to each ¹⁷⁷Lu-PSMA I&T infusion and the last visit of each cycle (weeks 4, 8, 12, 16, 20, 24 and 28) during the treatment period; and at EOT
Clinical assessment pre- and post 177Lu-PNT2002 administration
ECG Pre- and post-infusion (within 4 hr. post-dose) of all treatments with ¹⁷⁷Lu-PSMA I&T and at EOT Tumor marker PSA Week of first ¹⁷⁷Lu-PSMA I&T treatment, prior to infusion, and every 4 weeks during the treatment period, at the progression visit, at EOT, and if progression has not occurred by EOT, every 8 weeks until progression Hematology WBC count, WBC differential (3-part), RBC count, hemoglobin, hematocrit, mean corpuscular volume, platelet count Prior to ¹⁷⁷Lu-PSMA I&T infusions and every 2 weeks between infusions, at the progression visit and at EOT
Blood Chemistry Electrolytes (sodium, potassium, calcium, chloride, bicarbonate, phosphate, magnesium), total protein, serum creatinine, albumin, glucose (non-fasting), ALP, LDH, CK, total bilirubin, AST, ALT
Vital signs Blood pressure, heart rate, temperature, respiratory rate
Correlative samples (optional)
Circulating factors to predict response Baseline and Weeks 8, 16, 24, and EOT PK Blood and urine samples (up to 20 patients from selected sites)
Baseline Imaging CT/MRI chest, abdomen, pelvis, whole body bone scan Weeks 8, 16, 24, EOT, and every 8 weeks until progression
Toxicity AEs Every visit from first dose of ¹⁷⁷Lu-PSMA I&T through EOT SAEs Every visit from Screening through EOT
Late stage radiation toxicities If progression has not occurred by EOT, every 8 weeks until completion of LTFU

### Concomitant Medications

Review of concomitant medications: Every visit through EOT. If progression has not occurred by EOT, the use of anti-cancer therapy (although prohibited) will be collected every 8 weeks until progression.

### Investigations Timing

Opioid use Review of opioid medications Every visit until initiation of opioid use
Quality-of-life FACT-P Baseline and Weeks 8, 16, 24, and EOT
Pain assessment BPI-SF, AQA, review of skeletal events Day 1 and Weeks 4, 8, 12, 16, 20, 24 and 28 during the treatment period and at EOT
If progression has not occurred by EOT, AQA will be completed every 8 weeks until progression
¹⁷⁷Lu-PSMA I&T infusion will occur on the first day of each cycle, ending at the 4th cycle (maximum number of cycles). The first cycle starts at the first visit (Week 0) and occurs every 8 weeks thereafter.

### Control Arm Treatment

Patients randomized to Arm B will be treated with abiraterone or enzalutamide and will undergo the following investigations. Patients that progress in Arm B, will be assessed and, if eligible, crossover to receive ¹⁷⁷Lu-PSMA I&T.

### Control Arm Treatment

### Investigations Timing

Clinical assessment Limited physical examination, body weight measurement, vital signs, ECOG status
Prior to first control arm treatment, and every 4 weeks thereafter until EOT.
Tumor marker PSA Baseline (prior to receiving first control arm treatment) and every 4 weeks during the treatment period, at the progression visit, at EOT, and if progression has not occurred by EOT, every 8 weeks until progression
Hematology WBC count, 3-part differential, RBC count, hemoglobin, hematocrit, mean corpuscular volume, platelet count Baseline (prior to receiving first control arm treatment), every 4 weeks, at the progression visit and at EOT
Blood chemistry Electrolytes, serum creatinine, glucose (non-fasting), calcium, phosphate, magnesium, albumin, total protein, ALP, LDH, CK, bilirubin, ALT, AST
Vitals Blood pressure, heart rate, temperature, respiratory rate
Imaging CT/MRI chest, abdomen, pelvis, whole body bone scan
Weeks 8, 16, 24, EOT, and every 8 weeks until progression
Toxicity AEs Every visit from first dose of Investigational Product (i.e. PSMA Imaging Agent) through EOT(AE assessment should be every 2 weeks and can be done via phone call if no other procedures are required.
SAEs Every visit from Screening through EOT (SAE assessment should be every 2 weeks and can be done via phone call if no other procedures are required)
Concomitant medications Review of concomitant medications Every visit through EOT If progression has not occurred by EOT, the use of anti-cancer therapy (although prohibited) will be collected every 8 weeks until progression.
Opioid use Review of opioid medications Every visit until initiation of opioid use
Quality-of-life FACT-P Day 1 and Week 8, 16, 24 during the treatment period and EOT
Pain assessment BPI-SF, AQA, review of skeletal events Day 1 and Week 4, 8, 12, 16, 20, 24, and 28 during the treatment period, and at EOT
If progression has not occurred by EOT, AQA will be collected every 8 weeks until progression.
Treatment compliance Review of treatment compliance Baseline through EOT

### Progression Visit and Crossover

All patients who have shown progression (as described above) will undergo the following procedures, and all data will be recorded in the eCRF.
Assessment and recording of vital signs.
PSA measurement.
Assessment and recording of AE/SAEs.
Assessment and recording of concomitant medications, including opioid use.
Final assessment and recording of compliance with control arm treatment (Arm B).
Hematology.
Blood chemistry.
Time to second progression per Investigator assessment.

For patients in Arm A, continuing administration of subsequent ¹⁷⁷Lu-PSMA I&T infusions for patients who have met the criteria for individual progression (radiographic, biochemical, or clinical) will be determined in discussion between the investigator and the sponsor.

For patients in Arm B who are eligible to crossover as defined below will begin taking ¹⁷⁷Lu-PSMA I&T at Cycle 1, Week 0 in the SOA. However, ECGs and correlative samples are not required for these patients and imaging is reviewed locally. If the patient does not elect to crossover and has not completed the EOT visit, they should remain on their study schedule until 8 weeks after last dose, complete the EOT/Safety Follow-Up Visit, and then enter LTFU.

To determine eligibility for the crossover, patients in Arm B must undergo the following assessments. These can be done as part of their Progression visit. If it has been more than 30 days since their Progression visit, these should be repeated:
Final assessment and recording of compliance with control arm treatment (Arm B).
Vital signs.
Hematology, blood chemistry, and PSA.
AEs/SAEs, concomitant medication, including opioid use, and skeletal event review.
FACT-P and BPI-SF questionnaires.
AQA.
Tumor assessments (CT/MRI and whole body bone scans).
   In order to be eligible for this crossover, patients in Arm B must meet the following criteria:
Have experienced radiological progression on the control arm treatment by BICR as defined by RECIST 1.1 and PCWG3 of the protocol.
Have not started an alternative anti-cancer therapy.
Have no ongoing uncontrolled toxicities.
Adequate organ function, independent of transfusion:

### Bone Marrow Reserve:

WBC count≥2.5×109/L OR ANC≥1.5×109/L.

### Platelets≥100×109/L.

Hemoglobin≥5.6 g/dL.

### Liver Function:

Total bilirubin≤1.5×institutional ULN. For patients with known Gilbert's syndrome, ≤3×ULN is permitted.

### ALT or AST≤3.0×ULN.

### Renal Function:

Serum/plasma creatinine≤1.5×ULN or creatinine clearance≥50 mL/min.

### Albumin≥30 g/L.

If patients are not eligible for crossover, then they should instead complete the End-of-Study Visit and go into Long Term Follow-up.

### ¹⁸F-FDG PET (Optional)

¹⁸F-FDG PET CT/MRI imaging will be performed at Screening as per institutional standard procedures to assess for metabolic activity of detected lesions. This test is optional and will be performed at Screening only for patients that consent to this additional test.

### PSMA-PET

PSMA-PET imaging will be done within 30 days of randomization, but after all other eligibility assessments have been conducted.
PSMA imaging will be performed using ⁶⁸Ga-PSMA or ¹⁸F-DCFPyL as coordinated by the Sponsor. PET images will be reviewed centrally to identify PSMA-avid sites and quantify tracer uptake to determine eligibility and correlation with treatment response and/or progression.
High PSMA avidity determined by central review will be defined as:
For patients with measurable disease (per RECIST 1.1 criteria)
Threshold standard uptake value (SUV)max≥15 at one site of disease, and/or
SUVmax>10 at all measurable disease sites.
For those without measurable disease, at least 1 positive lesion SUVmax>10 must be evident.

### Progression

Radiographic progression should only be confirmed by BICR. For patients who show signs of biochemical or clinical progression, every effort should be made to confirm this progression with CT/MRI and whole body bone scans. In instances where there is disagreement between the investigator and central reader, the matter should be brought to the medical monitor for resolution.

Patients in any arm who discontinue due to progression will attend a Progression Visit. If a patient discontinues study treatment before confirmed BICR-assessed radiographic progression is declared, they should be followed for radiographic progression by BICR and OS as per the SOA.

Patients in Arm B who radiographically progress by BICR will be assessed and, if eligible, crossover to receive ¹⁷⁷Lu-PSMA I&T at Cycle 1 in the SOA.

### Radiographic Progression

Determination of radiographic progression will be performed by the BICR on the basis of conventional imaging.

### CT and MRI Tumor Assessment

CT or MRI images of the chest, abdomen, and pelvis will be used to assess for progression as per PCWG3-modified RECIST 1.1 criteria, with other areas of disease investigated based on the signs and symptoms of individual patients. All imaging performed following initiation of treatment (¹⁷⁷Lu-PSMA I&T or control arm) should include any suspected sites of new disease. Nodal and visceral disease progression will be determined as per PCWG3-modified RECIST 1.1 criteria: an increase of 20% or more in the target lesion SOD, in relation to the smallest sum observed on the study. Target lesion SOD must be increased by at least 5 mm.

### Bone Scans

Radiographic progression in bone lesions is to be assessed by bone scintigraphy performed as per institutional standard technique. Sites observed as positive on the bone scan should be considered significant, with unequivocal sites of malignant disease recorded as metastatic bone lesions.

Progression of bone disease will be determined as per PCWG3 criteria: the appearance of more than one new bone lesion on an on-treatment scan, with at least 2 additional new lesions seen on the next consecutive scan (confirmatory scan).

### Biochemical Progression

Biochemical progression will be determined as per PCWG3 criteria, as 2 sequential increasing measurements of PSA above the baseline, taken 1 week apart. PSA levels must be 2.0 ng/mL or higher. Patients should not be discontinued from treatment due to biochemical progression. In order to prevent early withdrawal from the treatment phase, PSA results will not be sent back to sites prior to an interim or final analysis.

### Unequivocal Clinical Progression

Clinical progression is defined as the lack of further benefit to the patient from ¹⁷⁷Lu-PSMA I&T treatment or control arm treatment alone and one or more of the following:
Initiation of chronic opioid use for new onset of prostate cancer pain suspected due to disease progression defined as daily use for more than 7 consecutive days or more than 10 days within a 14-day period, or
Immediate need to initiate of cytotoxic chemotherapy for prostate cancer, or
ECOG performance status deterioration to ≥3 due to prostate cancer, or
Radiation or surgical therapy for complications of prostate cancer tumor progression, excluding palliative radiotherapy (in treatment of pain at site of bone metastases present at baseline, unless indicative of disease progression).

Every attempt should be made to keep a patient on study treatment until radiographic progression by BICR is declared.

### Symptomatic Skeletal-Related Event

The occurrence of one or more of the following should be considered a symptomatic skeletal related event (SSRE):
Use of radiation therapy to prevent or relieve skeletal symptoms.
Occurrence of new symptomatic pathological bone fractures (vertebral or non-vertebral). Radiologic documentation is required.
Occurrence of spinal cord compression. Radiologic documentation required. Orthopedic surgical intervention for bone metastasis.

### PSA Measurements and Response

Collection of blood samples for PSA assessment will take place during the treatment period.

Quantification of blood PSA levels will be performed by a central laboratory, with the results blinded to the site.

A patient will be regarded as having a single PSA visit response, if their PSA level at any post-dose visit is reduced by 50% or more compared with baseline.

A patient will be regarded as having a confirmed PSA response if they have a reduction in PSA level of 50% or more compared with baseline that is confirmed at the next assessment at least 3 weeks later (i.e., decrease relative to baseline of at least 50% documented on 2 consecutive occasions at least 3 weeks apart).

### ECOG Assessment

The investigator or his delegate will assess the patient's performance status using the ECOG Scale of Performance Status. The ECOG scale evaluates the patient's functioning in terms of their ability for self-care, daily activities, and physical activity.

### Grade ECOG Performance Status

0 Fully active, able to carry on all pre-disease performance without restriction
1 Restricted in physically strenuous activity but ambulatory and able to carry out work of a light or sedentary nature, e.g., light housework, office work
2 Ambulatory and capable of all self-care but unable to carry out any work activities; up and about more than 50% of walking hours
3 Capable of only limited self-care; confined to bed or chair more than 50% of waking hours
4 Completely disabled; cannot carry on any self-care; totally confined to bed or chair
5 Dead

### PSA Response

PSA response is defined as the proportion of patients achieving a ≥50% decrease in PSA from baseline to the lowest post-baseline PSA result, confirmed by a second consecutive PSA assessment at least 3 weeks later.
A patient will be regarded as having a single PSA visit response if their PSA level at any post-dose visit is reduced by 50% or more compared with baseline.
A patient will be regarded as having a confirmed PSA response if they have a reduction in PSA level of 50% or more compared with baseline that is confirmed at the next assessment at least 3 weeks later (i.e., decrease relative to baseline of at least 50% documented on 2 consecutive occasions at least 3 weeks apart).

The number and percentage of PSA responders will be presented by treatment group. A 95% confidence interval for the difference of proportions of responders between the two treatment groups will be displayed.

### Biochemical Progression-Free Survival

Defined as time from the date of randomization to the date of the first PSA increase from baseline≥25% and ≥2 ng/mL above nadir confirmed by a second PSA measurement defining progression≥3 weeks later per PCWG3, or death from any cause in the absence of progression.

Patients who do not progress or die including those who withdraw from the study or are lost to follow-up will be censored at the date of last valid PSA measurement from a scheduled or unscheduled visit.

bPFS will be described using Kaplan-Meier estimates with 95% CIs. Summaries will also include the number at risk, the number of patients with events, and the number of patients censored.

### Example 3: Preliminary Clinical Data

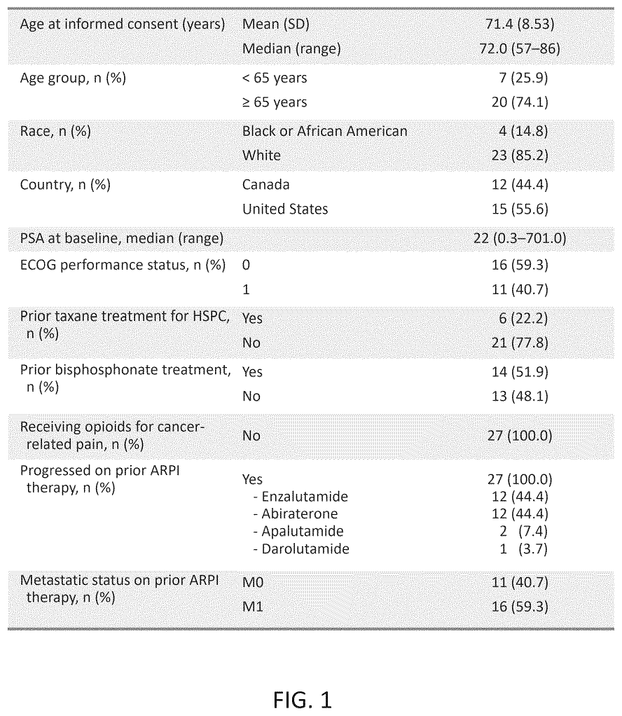

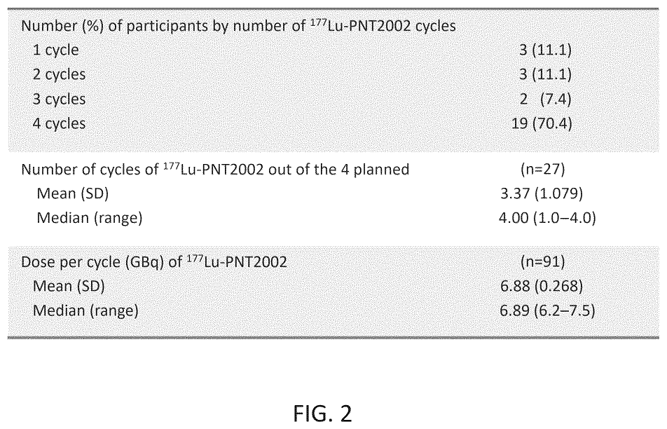

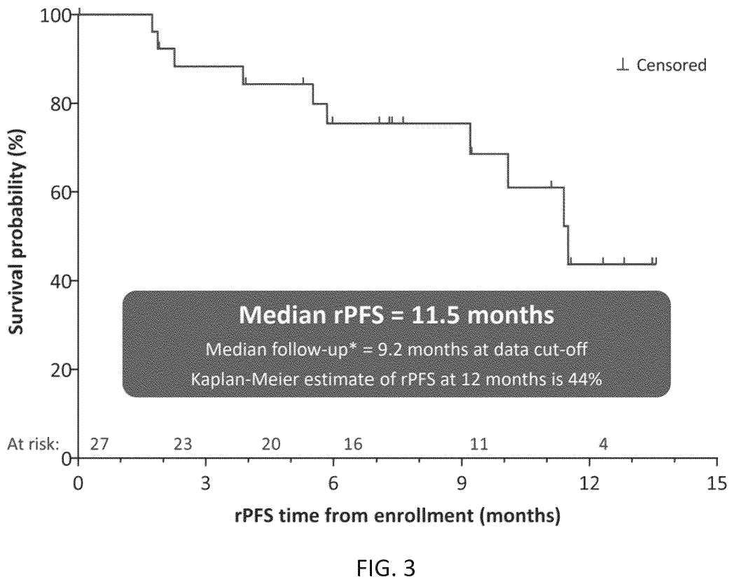

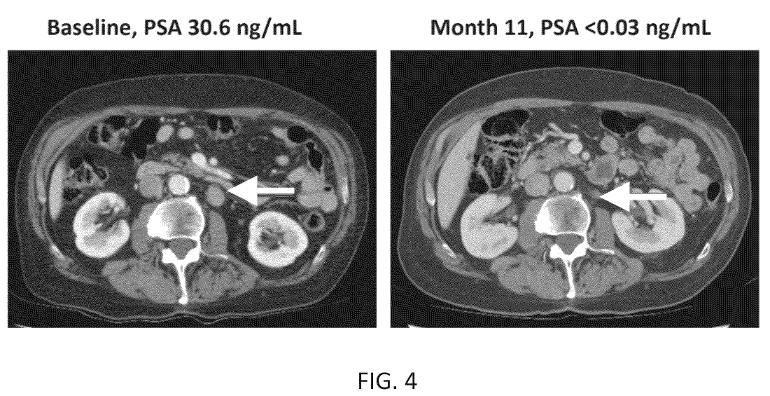

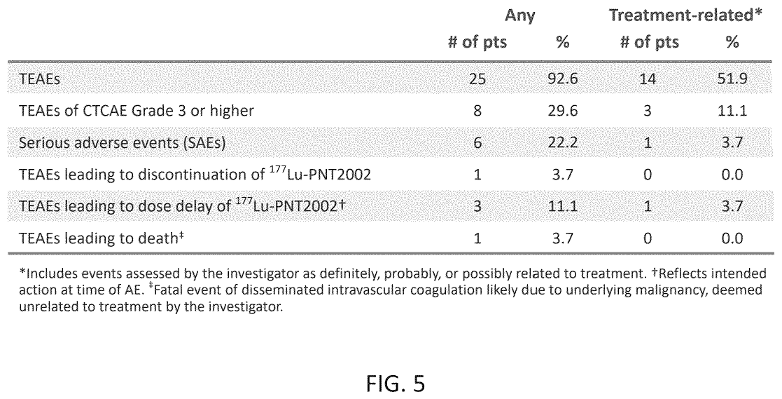

SPLASH (NCT04647526) is a multinational, phase III, open-label, randomized study to evaluate efficacy and safety of ¹⁷⁷Lu-PSMA I&T (177Lu-PNT2002), following the protocols set forth in Example 1 above, in metastatic castration-resistant prostate cancer (mCRPC) after androgen receptor pathway inhibitor (ARPI) therapy. SPLASH is designed to evaluate radioligand therapy earlier in the treatment pathway and using fewer and lower doses, as compared to the currently approved indication for radioligand treatment in prostate cancer.
(i) SPLASH dosimetry results: The average dose to red marrow was 0.034 Gy/GBq, well below critical thresholds, enabling a potential opportunity for combination therapy. Organs receiving the largest absorbed doses were the lacrimal glands at 1.2 Gy/GBq, followed by the kidneys at 0.73 Gy/GBq.
(ii) Preliminary safety and efficacy results from the dosimetry lead-in sub-study of SPLASH: The SPLASH study commenced with a 27-participant dosimetry and safety lead-in phase prior to randomization expansion. See FIG. 1. Participants were enrolled with tumours exhibiting high PSMA uptake on PET/CT per blinded independent central review, chemotherapy naïve for CRPC, progressing on an ARPI, and adequate bone marrow and end organ reserve. All participants received up to four cycles of 177Lu-PNT2002 at 6.8 GBq per cycle every 8 weeks. See FIG. 2. This dose was selected to minimize long-term risk while providing a dose found to be efficacious in previous studies with 177Lu-PSMA-I&T.
   In the SPLASH lead-in cohort, median rPFS time was 11.5 months, as compared to the control arm benchmarks of 3.5-4.2 months for individuals with progressive mCRPC post-ARPI failure receiving similar treatment. See FIG. 3. A radiographic objective response (CR, PR) was achieved in 60% of the 10 participants with evaluable disease at baseline. FIG. 4 shows a complete response participant vignette. Retroperitoneal lymph node target lesion (indicated by white arrows) with a short axis measurement of 2.1 cm at baseline, and the complete response of the lesion imaged at month 11.
   ¹⁷⁷Lu-PSMA I&T (177Lu-PNT2002) was well tolerated with no treatment-related deaths and few treatment-related AEs of grade 3 or higher. See FIG. 5.

### List of Abbreviations and Terms Used in Examples

177Lu Lutetium-177
ADT Androgen deprivation therapy
AE Adverse event
AESI Adverse event of special interest
ALP Alkaline phosphatase
ALT Alanine aminotransferase
ANC Absolute neutrophil count
AQA Analgesic quantification algorithm
ARAT Androgen receptor axis targeted
ART Activity reference time
AST Aspartate aminotransferase
BICR Blinded independent central review
bPFS Biochemical progression-free survival
BPI-SF Brief Pain Inventory-Short Form
CI Confidence interval
CMH Cochran-Mantel-Haenszel
CR Complete response
CRPC Castration-resistant prostate cancer
CT Computed tomography
CTCAE Common Terminology Criteria for Adverse Events
DCO Discontinuation
DOR Duration of response
DMC Data Monitoring Committee
DSMC Data Safety Monitoring Committee
ECG Electrocardiography
ECOG Eastern Cooperative Oncology Group
eCRF Electronic case report form
EQ-5D-5L EuroQol 5-dimension, 5-level health state utility index
EOT End of treatment
FACT-P Functional assessment of cancer therapy-prostate cancer
FDA Food and Drug Administration
FDG-PET Fluorodeoxyglucose-positron emission tomography
GBq Gigabecquerel
GCP Good clinical practice
GCP II Glutamate carboxypeptidase II
Gy Gray
HDL High-density lipoprotein
HR Hazard ratio
HRQoL Health-related quality of life
HSPC Hormone sensitive prostate cancer
ICH International Conference on Harmonization
IRB Institutional Review Board
ITT Intended-to-treat
IV Intravenous infusion
IVRS Interactive voice response system
IWRS Interactive web response system
LDL Low-density lipoprotein
LHRH Luteinizing hormone-releasing hormone
MBq Megabecquerel
MGy Megagray
mCRPC Metastatic castrate-resistant prostate cancer
MRI Magnetic resonance imaging
NAALADase I N-acetyl-L-aspartyl-L-glutamate peptidase I
OAE Other adverse events
ORR Objective response rate
OS Overall survival
PARP Poly ADP ribose polymerase
PCA Prostate cancer
PCWG3 Prostate Cancer Working Group 3
PD Progressive disease
PET Positron emission tomography
PFS Progression-free survival
PK Pharmacokinetics
PP Per protocol
PR Partial response
PSA Prostate specific antigen
PSMA Prostate-specific membrane antigen
rPFS Radiological progression-free survival
QoL Quality of life
REB Research Ethics Board
RECIST Response evaluation criteria in solid tumors
SAE Serious adverse event
SAP Statistical analysis plan
SD Stable disease
SOA Schedule of activities
SOC Standard of care
SOD Sum of the diameter
SSRE Symptomatic skeletal-related event
SOP Standard operating procedure
SPECT Single-photon emission computed tomography
SUV Standard uptake value
TOC Time of calibration
ULN Upper limit of normal
USPI United States package insert
WBC White blood cell count REFERENCES FOR EXAMPLES
1. Tannock I F, de Wit R, Berry W R, et al. Docetaxel plus prednisone or mitoxantrone plus prednisone for advanced prostate cancer. N Engl J Med. 2004; 351(15):1502-1512.
2. Canadian Cancer Society's Advisory Committee on Cancer Statistics. Canadian Cancer Statistics 2016. Toronto, ON: Canadian Cancer Society; 2016.
3. Siegel R L, Miller K D, Jemal A. Cancer statistics, 2017. CA Cancer J Clin. 2017; 67(1):7-30.
4. Scher H I, Solo K, Valant J, Todd M B, Mehra M. Prevalence of prostate cancer clinical states and mortality in the United States: estimates using a dynamic progression model. PLoS One. 2015; 10(10):e0139440. Published 2015 Oct. 13.
5. Smith M R, Saad F, Chowdhury S, et al. Apalutamide treatment and metastasis-free survival in prostate cancer. N Engl J Med. 2018; 378(15):1408-1418.
6. Fizazi K, Shore N, Tammela T L, et al. Darolutamide in nonmetastatic, castrationresistant prostate cancer. N Engl J Med. 2019; 380(13):1235-1246.
7. de Bono J S, Logothetis C J, Molina A, et al. Abiraterone and increased survival in metastatic prostate cancer. N Engl J Med. 2011; 364(21):1995-2005.
8. Ryan C J, Smith M R, Fizazi K, et al. Abiraterone acetate plus prednisone versus placebo plus prednisone in chemotherapy-naïve men with metastatic castration-resistant prostate cancer (COU-AA-302): final overall survival analysis of a randomized, double-blind, placebo-controlled phase 3 study. Lancet Oncol. 2015; 16( ):152-160.
9. Scher H I, Fizazi K, Saad F, et al. Increased survival with enzalutamide in prostate cancer after chemotherapy. N Engl J Med. 2012; 367(13):1187-1197.
10. Fizazi K, Scher H I, Molina A, et al. Abiraterone acetate for treatment of metastatic castration-resistant prostate cancer: final overall survival analysis of the COU-AA-301 randomised, double-blind, placebo-controlled phase 3 study. Lancet Oncol. 2012; 13(10):983-992.
11. Shivji A, Ali R, North S, Sawyer M, Ghosh S, Chambers C. Real world evidence: Abiraterone use post-docetaxel in metastatic castrate-resistant prostate cancer. J Oncol Pharm Pract. 2019; 25(6):1293-1300.
12. Ryan C J, Smith M R, de Bono J S, et al. Abiraterone in metastatic prostate cancer without previous chemotherapy. N Engl J Med. 2013; 368(2):138-148.
13. Beer T_{M}, Armstrong A J, Rathkopf D E, et al. Enzalutamide in metastatic prostate cancer before chemotherapy. N Engl J Med. 2014; 317:424-433.
14. Bauman G, Charette M, Reid R, Sathya J. Radiopharmaceuticals for the palliation of painful bone metastasis—a systemic review. Radiother Oncol. 2005; 75(3):258-270.
15. Parker C, Nilsson S, Heinrich D, et al. Alpha emitter radium-223 and survival in metastatic prostate cancer. N Engl J Med. 2013; 369(3):213-223.
16. Silver D A, Pellicer I, Fair W R, Heston W D, Cordon-Cardo C. Prostate-specific membrane antigen expression in normal and malignant human tissues. Clin Cancer Res. 1997; 3(1):81-85.
17. Kasperzyk J L, Finn S P, Flavin R, et al. Prostate-specific membrane antigen protein expression in tumor tissue and risk of lethal prostate cancer. Cancer Epidemiol Biomarkers Prev. 2013; 22(12):2354-2363.
18. Meller B, Bremmer F, Sahlmann C O, et al. Alterations in androgen deprivation enhanced prostate-specific membrane antigen (PSMA) expression in prostate cancer cells as a target for diagnostics and therapy. EJNMMI Res. 2015; 5(1):66.
19. Tasch J, Gong M, Sadelain M, Heston W D. A unique folate hydrolase, prostate-specific membrane antigen (PSMA): a target for immunotherapy Crit Rev Immunol. 2001; 21(1-3):249-261.
20. Sokoloff M H, Yang X J, Fumo M, Mhoon D, Brendler C B. Characterizing prostatic adenocarcinomas in men with a serum prostate specific antigen level of <4.0 ng/mL. BJU Int. 2004; 93(4):499-502.
21. Hillier S M, Maresca K P, Femia F J, et al. Preclinical evaluation of novel glutamate-urealysine analogues that target prostate-specific membrane antigen as molecular imaging pharmaceuticals for prostate cancer. Cancer Res. 2009; 69(17):6932-6940.
22. Chen Y, Pullambhatla M, Foss C A, et al. 2-(3-{1-carboxy-5-[(6-[18F]fluoro-pyridine-3-carbonyl)-amino]-pentyl}-ureido)-pentanedioic acid, [18F]DCFPyL, a PSMA-based PET imaging agent for prostate cancer. Clin Cancer Res. 2011; 17(24):7645-7653.
23. Afshar-Oromieh A, Haberkorn U, Eder M, Eisenhut M, Zechmann C M. [68Ga]Galliumlabelled PSMA ligand as superior PET tracer for the diagnosis of prostate cancer: comparison with 18F-FECH. Eur J Nucl Med Mol Imaging. 2012; 39(6):1085-1086.
24. Maurer T, Gschwend J E, Rauscher I, et al. Diagnostic efficacy of (68)gallium-PSMA Positron emission tomography compared to conventional imaging in lymph node staging of 130 consecutive patients with intermediate to high risk prostate cancer. J Urol. 2016; 195(5):1436-1443.
25. Perera M, Papa N, Christidis D, et al. Sensitivity, specificity, and predictors of positive 68Ga-prostate-specific membrane antigen positron emission tomography in advanced prostate cancer: a systematic review and meta-analysis. Eur Urol. 2016; 70(6):926-937.
26. Eiber M, Maurer T, Souvatzoglou M, et al. Evaluation of hybrid 68Ga-PSMA ligand PET/CT in 248 patients with biochemical recurrence after radical prostatectomy. J Nucl Med. 2015; 56(5):668-674.
27. Dietlein M, Kobe C, Kuhnert G, et al. Comparison of [(18)F]DCFPyL and [(68)Ga]Ga-PSMA-HBED-CC for PSMA-PET imaging in patients with relapsed prostate cancer. Mol Imaging Biol. 2015; 17(4):575-584.
28. Ferreira G, Iravani A, Hofman M S, Hicks R J. Intra-individual comparison of 68Ga-PSMA-11 and 18F-DCFPyL normal-organ biodistribution. Cancer Imaging. 2019; 19(1):23.
29. Giesel F L, Will L, Lawal I, et al. Intraindividual comparison of 18F-PSMA-1007 and 18F-DCFPyL PET/CT in the prospective evaluation of patients with newly diagnosed prostate carcinoma: a pilot study. J Nucl Med. 2018; 59(7):1076-1080.
30. Bander N H, Milowsky M I, Nanus D M, Kostakoglu L, Vallabhajosula S, Goldsmith S J. Phase I trial of 177lutetium-labeled J591, a monoclonal antibody to prostate-specific membrane antigen, in patients with androgen-independent prostate cancer. J Clin Oncol. 2005; 23(21):4591-4601.
31. Tagawa S T, Milowsky M I, Morris M, et al. Phase II study of Lutetium-177-labeled antiprostate-specific membrane antigen monoclonal antibody J591 for metastatic castrationresistant prostate cancer. Clin Cancer Res. 2013; 19(18):5182-5191.
32. Benešová M, Scháfer M, Bauder-Wüst U, et al. Preclinical evaluation of a tailor-made DOTA-conjugated PSMA inhibitor with optimized linker moiety for imaging and endoradiotherapy of prostate cancer. J Nucl Med. 2015; 56(6):914-920.
33. Kratochwil C, Giesel F L, Eder M, et al. [177Lu]Lutetium-labelled PSMA ligand-induced remission in a patient with metastatic prostate cancer. Eur J Nucl Med Mol Imaging. 2015; 42(6):987-988.
34. Schlenkhoff C D, Knüpfer E, Essler M, Ahmadzadehfar H. Metastatic prostate cancer with restored hormone-response after radioligand therapy with 177Lu-PSMA-617. Clin Nucl Med. 2016; 41(7):572-573.
35. Roll W, Bode A, Weckesser M, Bogemann M, Rahbar K. Excellent response to 177Lu-PSMA-617 radioligand therapy in a patient with advanced metastatic castration resistant prostate cancer evaluated by 68Ga-PSMA PET/CT. Clin Nucl Med. 2017; 42(2):152-153.
36. Ahmadzadehfar H, Eppard E, Kürpig S, et al. Therapeutic response and side effects of repeated radioligand therapy with 177Lu-PSMA-DKFZ-617 of castrate-resistant metastatic prostate cancer. Oncotarget. 2016; 7(11):12477-12488.
37. Kratochwil C, Giesel F L, Stefanova M, et al. PSMA-targeted radionuclide therapy of metastatic castration-resistant prostate cancer with 177Lu-labeled PSMA-617. J Nucl Med. 2016; 57(8):1170-1176.
38. Rahbar K, Bode A, Weckesser M, et al. Radioligand therapy with 177Lu-PSMA-617 as a novel therapeutic option in patients with metastatic castration resistant prostate cancer. Clin Nucl Med. 2016; 41(7):522-528.
39. Rahbar K, Schmidt M, Heinzel A, et al. Response and tolerability of a single dose of 177Lu-PSMA-617 in patients with metastatic castration-resistant prostate cancer: a multicenter retrospective analysis. J Nucl Med. 2016; 57(9):1334-1338.
40. Fendler W P, Reinhardt S, Ilhan H, et al. Preliminary experience with dosimetry, response and patient reported outcome after 177Lu-PSMA-617 therapy for metastatic castration resistant prostate cancer. Oncotarget. 2017; 8(2):3581-3590.
41. Rahbar K, Ahmadzadehfar H, Kratochwil C, et al. German multicenter study investigating 177Lu-PSMA-617 radioligand therapy in advanced prostate cancer patients. J Nucl Med. 2017; 58(1):85-90.
42. Weineisen M, Simecek J, Schottelius M, Schwaiger M, Wester H J. Synthesis and preclinical evaluation of DOTAGA-conjugated PSMA ligands for functional imaging and endoradiotherapy of prostate cancer. EJNMMI Res. 2014; 4(1):63.
43. Weineisen M, Schottelius M, Simecek J, et al. 68Ga- and 177Lu-labeled PSMA I&T: optimization of a PSMA-targeted theranostic concept and first proof-of-concept human studies. J Nucl Med. 2015; 56(8):1169-1176.
44. Heck M M, Retz M, D'Alessandria C, et al. Systemic radioligand therapy with (177)Lu labeled prostate specific membrane antigen ligand for imaging and therapy in patients with metastatic castration resistant prostate cancer. J Urol. 2016; 196(2):382-391.
45. Heck M M, Tauber M, Schwaiger S, et al. Treatment outcome, toxicity, and predictive factors for radioligand therapy with 177Lu-PSMA-I&T in metastatic castration-resistant prostate cancer. Eur Urol. 2019; 75(6):920-926.
46. Baum R P, Kulkarni H R, Schuchardt C, et al. 177Lu-labeled prostate-specific membrane antigen radioligand therapy of metastatic castration-resistant prostate cancer: safety and efficacy. J Nucl Med. 2016; 57(7):1006-1013.
47. Kulkarni H, Schuchardt C, Singh A, Langbein T, Baum R. Early initiation of Lu-177 PSMA radioligand therapy prolongs overall survival in metastatic prostate cancer. J Nucl Med. 2018; 59(suppl 1):529.
48. Acar E, Özdoǧan Ö, Aksu A, Derebek E, Bekis R, Capa Kaya G. The use of molecular volumetric parameters for the evaluation of Lu-177 PSMA I&T therapy response and survival. Ann Nucl Med. 2019; 33(9):681-688.
49. Yamoah K, Showalter T N, Ohri N. Radiation therapy intensification for solid tumors: a systematic review of randomized trials. Int J Radiat Oncol Biol Phys. 2015; 93(4):737-745.
50. Marin G, Vanderlinden B, Karfis I, et al. Accuracy and precision assessment for activity quantification in individualized dosimetry of 177Lu-DOTATATE therapy. EJNMMI Phys. 2017; 4(1):7.
51. Chung D Y, Kang D H, Kim J W, et al. Comparison of oncologic outcomes between two alternative sequences with abiraterone acetate and enzalutamide in patients with metastatic castration-resistant prostate cancer: a systematic review and meta-analysis. Cancers (Basel). 2019; 12(1):8.
52. De Bono J, Mateo J, Fizazi K, et al. Olaparib for metastatic castration-resistant prostate cancer. N Engl J Med. 2020; 382(22):2091-2102.
53. Lorente D, Omlin A, Ferraldeschi R, et al. Tumour responses following a steroid switch from prednisone to dexamethasone in castration-resistant prostate cancer patients progressing on abiraterone. Br J Cancer. 2014; 111(12):2248-2253.
54. Okamoto S, Thieme A, Allman J, et al. Radiation dosimetry for 177Lu-PSMA I&T in metastatic castration-resistant prostate cancer: absorbed dose in normal organs and tumor lesions. J Nucl Med. 2017; 58(3):445-450.
55. Dawson L A, Kavanagh B D, Paulino A C, et al. Radiation-associated kidney injury. Int J Radiat Oncol Biol Phys. 2010; 76(3 Suppl):S108-S115.
56. Emami B, Lyman J, Brown A, et al. Tolerance of normal tissue to therapeutic irradiation. Int J Radiat Oncol Biol Phys. 1991; 21(1):109-122.
57. Wu V W C, Leung K Y. A review on the assessment of radiation induced salivary gland damage after radiotherapy. Front Oncol. 2019; 9:1090.
58. Kesavan M, Turner J H, Meyrick D, Yeo S, Cardaci G, Lenzo N P. Salvage therapy of advanced castrate-resistant prostate cancer with lutetium-177-labeled prostate-specific membrane antigen: efficacy and safety in routine practice. Cancer Biother Radiopharm. 2018; 33(7):274-281.
59. Gafita A, Kroenke M, Tauber R, Weber W, Eiber M. Correlation of 12-week PSA response after 177Lu-PDMA radioligand therapy, baseline tumor burden and overall survival in mCRPC. J Nucl Med. 2019; 60:78.
60. Siegel J A, Thomas S R, Stubbs J B, Stabin M G, Hays M T, Koral K F, et al. MIRD pamphlet no. 16: Techniques for quantitative radiopharmaceutical biodistribution data acquisition and analysis for use in human radiation dose estimates. J Nucl Med. 1999 February; 40(2):37S-61S.
61. Siegel J A. Establishing a Clinically Meaningful Predictive Model of Hematologic Toxicity in Nonmyeloablative Targeted Radiotherapy: Practical Aspects and Limitations of Red Marrow Dosimetry. Cancer Biotherapy & Radiopharmaceuticals. 2005; 20(2):126-140.
62. Ferrer L, Kraeber-Bodéré F, Bodet-Milin C, et al. Three methods assessing red marrow dosimetry in lymphoma patients treated with radioimmunotherapy. Cancer. 2010; 116(4 Suppl):1093-100.
63. Forrer F, Krenning E P, Kooij P P, et al. Bone marrow dosimetry in peptide receptor radionuclide therapy with [177Lu-DOTA(0),Tyr(3)]octreotate. Eur J Nucl Med Mol Imaging. 2009; 36(7):1138-46.
64. Herrmann K, Bluemel C, Weineisen M et al. Biodistribution and radiation dosimetry for a probe targeting prostate-specific membrane antigen for imaging and therapy. J Nucl Med. 2015; 56(6):855-61.
65. Sgouros G. Bone marrow dosimetry for radioimmunotherapy: theoretical considerations. J Nucl Med. 1993; 34:689-94.
66. Stabin M G, Sparks R B, Crowe E B. OLINDA/EXM—The Second Generation Personal Computer Software For Internal Dose Assessment In Nuclear Medicine. J Nucl Med. 2005; (46):1023-1027.
67. Stabin M, Farmer A. OLINDA/EXM 2.0: The new generation dosimetry modeling code. J Nucl Med. 2012; 53(suppl 1):585.
68. Thang S P, Violet J, Sandhu S, et al. Poor Outcomes for Patients with Metastatic Castration-resistant Prostate Cancer with Low Prostate-specific Membrane Antigen (PSMA) Expression Deemed Ineligible for 177Lu-labelled PSMA Radioligand Therapy. Eur Urol Oncol. 2019; 2(6):670-676.
69. Emmett L, Crumbaker M, Ho B, et al. Results of a Prospective Phase II Pilot Trial of 177Lu-PSMA-617 Therapy for Metastatic Castration Resistant Prostate Cancer Including Imaging Predictors of Treatment Response and Patterns of Progression. Clin Genitourin Cancer. 2019; 17(1):15-22.
70. Hofman M S, Emmet L, Violet J et al. TheraP: a randomized phase 2 trial of 177Lu-PSMA-617 theranostic treatment vs cabazitaxel in progressive metastatic castrationresistant prostate cancer (Clinical Trial Protocol ANZUP 1603). BJU Int. 2019; 124(Supplement 1):5-13.
71. Buckley O, O'Keffe S, Geoghegan T, et al. 99mTc bone scintigraphy superscans: a review. Nucl Med Commun. 2007; 28(7)521-527.
72. Scher H I, Morris M J, Stadler W M, et al. Trial design and objectives for castration resistant prostate cancer: updated recommendations from the Prostate Cancer Clinical Trials Working Group 3. J Clin Oncol. 2016; 34(12):1402-1418.
73. Shen S, DeNardo G L, Sgouros G, O'Donnell R T, DeNardo S J. Practical determination of patient-specific marrow dose using radioactivity concentration in blood and body. J Nucl Med. 1999; 40(12):2102-2106.
74. de Wit R, de Bono J, Sternberg C N, et al. Cabazitaxel versus Abiraterone or Enzalutamide in Metastatic Prostate Cancer. N Engl J Med. 2019; 381(26):2506-2518.
75. FDA guidance on Clinical Trial Endpoints for the Approval of Cancer Drugs and Biologics. Available from https://www.fda.gov/downloads/Drugs/Guidances/ucm071590.pdf.
76. Chung K C, Barlev A, Braun A H, et al. Assessing analgesic use in patients with advanced cancer: development of a new scale—the Analgesic Quantification Algorithm. Pain Medicine. 2014; 15:225-232.
77. Erwin W. A novel heart-contents image-based Yttrium-90-Zevalin red marrow residence time estimator. JNM May 2010, 51 (supplement 2) 1323.

### INCORPORATION BY REFERENCE

The entire contents of all patents, published patent applications and other disclosures cited herein are hereby expressly incorporated herein in their entireties by reference.

While this invention has been particularly shown and described with references to preferred embodiments thereof, it will be understood by those skilled in the art that various changes in form and details may be made therein without departing from the scope of the invention encompassed by the appended claims.

1. A method for treating a subject suffering from prostate cancer, wherein the subject (i) has exhibited prostate cancer progression during or after treatment with one or more androgen receptor inhibitor agents and (ii) is chemotherapy-naïve before treatment with the one or more androgen receptor inhibitor agents; said method comprising:
administering to the subject an amount of a complex of lutetium-177 and EuK-Sub-kf-iodo-y-DOTAGA providing from a 4 to 10 Gbq dose at least four times, at four week or longer intervals between doses.

2. (canceled)

3. The method of claim 1 wherein the subject has exhibited cancer progression during treatment with one or more of abiraterone, enzalutamide, apalutamide, darolutamide, cimetidine, orteronel, galeterone, seviteronel, topilutamide, bicalutamide, fluamide and/or nilutamide.

4. (canceled)

5. (canceled)

6. The method of claim 1 wherein the subject has exhibited cancer progression following at least two months of treatment with one or more androgen receptor inhibitor agents.

7. The method of claim 1 wherein treatment with one or more androgen receptor inhibitor agents is the only chemotherapy administered to the subject before administering the lutetium-177 labeled PSMA-targeted radioligand.

8. The method of claim 1 wherein the subject is taxane chemotherapy-naïve and/or taxane chemotherapy adverse.

9. The method of claim 1 wherein the subject exhibits elevated PSMA expression relative to a healthy subject.

10. (canceled)

11. The method of claim 1 wherein the subject exhibits PSMA expression standard uptake value (SUV)max≥15 at one site of disease and/or SUVmax>10 at all measurable disease sites.

12. The method of claim 1 wherein the subject exhibits at least 1 positive lesion SUVmax≥10.

13. The method of claim 1 wherein the subject exhibits elevated PSA levels.

14. (canceled)

15. (canceled)

16. The method of claim 1 wherein administration of one or more androgen receptor inhibitor agents is terminated before administering the complex of lutetium-177 and EuK-Sub-kf-iodo-y-DOTAGA.

17. (canceled)

18. (canceled)

19. The method of claim 1 wherein the subject is suffering from metastatic castration-resistant prostate cancer (mCRPC).

20. The method of claim 19 wherein the subject is suffering from metastatic castration-resistant prostate cancer (mCRPC) with prostate-specific membrane antigen (PSMA)-avid lesions.

21. The method of claim 1 wherein the complex of lutetium-177 and EuK-Sub-kf-iodo-y-DOTAGA is administered in combination with one or more distinct chemotherapeutic agents.

22. The method of claim 1 wherein the complex of lutetium-177 and EuK-Sub-kf-iodo-y-DOTAGA comprises the following structure:

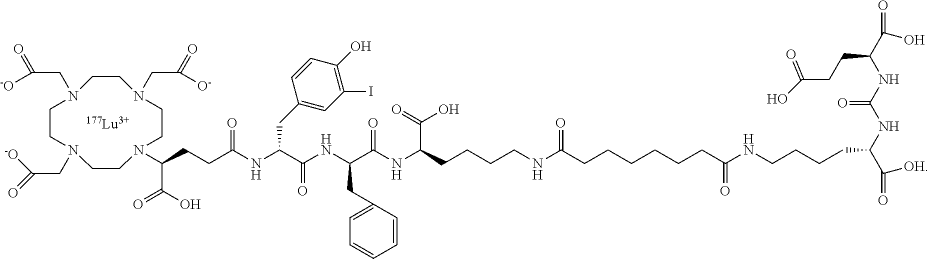

23. (canceled)

24. (canceled)

25. (canceled)

26. A kit, comprising:
a pharmaceutical preparation of a complex of lutetium-177 and EuK-Sub-kf-iodo-y-DOTAGA suitable for intravenous administration to a subject, and in an amount sufficient to provide from a 4 to 10 Gbq dose; and
a package insert containing instructions for use of the agent in the treatment of a subject exhibiting prostate-specific membrane antigen (PSMA)-positive metastatic castration-resistant prostate cancer (mCRPC) who has previously been treated with androgen receptor (AR) pathway inhibition but which subject is taxane chemotherapy-naïve or taxane chemotherapy adverse.

27. (canceled)

28. (canceled)

29. (canceled)

30. (canceled)

31. (canceled)

32. The kit of claim 26, wherein the lutetium-177 EuK-Sub-kf-iodo-y-DOTAGA complex is provided in a single dose form sufficient to provide 6.8 GBq+/−10% within shelf-life.

33. (canceled)

34. The kit of claim 26, wherein the pharmaceutical preparation includes Sodium Ascorbate at a concentration of 55-75 mg/mL and Sodium Gentisate at a concentration of 16-36 mg/mL and has a pH in the range of 5.0-7.0.

35. The kit of claim 26, wherein the pharmaceutical preparation has a radioactive concentration (RAC) of 0.5 to 1.2 GBq/mL at end of synthesis and formulation of the pharmaceutical preparation.

36. The kit of claim 26, wherein the pharmaceutical preparation has a total peptide content of less than or equal to 250 μg (sum of EuK-Sub-kf-iodo-y-DOTAGA and lutetium-177 EuK-Sub-kf-iodo-y-DOTAGA complex).

37. The kit of claim 26, wherein the pharmaceutical preparation is provided in a single dosage vial.

38. The kit of claim 26, wherein the pharmaceutical preparation is provided in a disposable syringe with syringe shield for handheld infusion administration.

39. The kit of claim 26, wherein the kit includes a radiation shielded pig in which the pharmaceutical preparation is transported in the packaged kit.

40. (canceled)

41. (canceled)
